(19) United States
(12) Patent Application Publication
(10) Pub. No.: US 2026/0137438 A1
(43) Pub. Date: May 21, 2026

(54) PULSED ELECTRIC FIELD ENERGY DELIVERY TIMING OPTIMIZATION

(71) Applicant: Galvanize Therapeutics, Inc., Redwood City, CA (US)

(72) Inventors: David W. HUNTER, Redwood City, CA (US); Robert E. NEAL, II, Redwood City, CA (US); Jacob I. LAUGHNER, Redwood City, CA (US); William S. KRIMSKY, Redwood City, CA (US)

(73) Assignee: Galvanize Therapeutics, Inc., Redwood City, CA (US)

(21) Appl. No.: 19/331,666

(22) Filed: Sep. 17, 2025

Related U.S. Application Data
(63) Continuation of application No. PCT/US24/20309, filed on Mar. 15, 2024.
(60) Provisional application No. 63/491,025, filed on Mar. 17, 2023.

(51) Int. Cl. A61B 18/12; A61B 18/00

(52) U.S. Cl. CPC A61B 18/1206; A61B 2018/00267; A61B 2018/00351; A61B 2018/00541; A61B 2018/00648; A61B 2018/00732; A61B 2018/0075; A61B 2018/00767; A61B 2018/00839; A61B 2018/126

(57) ABSTRACT

Devices, systems and methods are provided for treating target tissue with pulsed electric field energy in a manner that does not induce cardiac arrhythmias such as fibrillation of the heart and therefore negates any need for cardiac synchronization of energy delivery. Cardiac synchronization is typically employed to avoid delivering energy during portions of the cardiac cycle that are vulnerable to inducing fibrillation. Thus, herein parameters are manipulated to reduce the probability of inducing an arrhythmia. Typically, high voltage energy can trigger a premature action potential within the cardiac muscle as the delivered energy increases the cardiac muscle cell membrane permeability allowing ion transport, which can induce cardiac arrhythmias, especially ventricular fibrillation. However, both shorter pulse durations (i.e. higher frequencies such as 100-600 kHz) and biphasic waveform shapes theoretically and practically reduce the probability of inducing an arrhythmia.

### CROSS REFERENCE TO RELATED APPLICATIONS

This application is a continuation of PCT No. PCT/US24/020309, filed Mar. 15, 2024, which claims priority to and the benefit of U.S. Provisional Patent Application No. 63/491,025, filed Mar. 17, 2023, the disclosures of which are fully incorporated herein by reference.

### BACKGROUND

Pulsed electric field (PEF) therapies use brief applications of electrical energy to the body for the treatment of diseases and afflictions. In some instances, such delivery is to tissues within a body lumen, passageway or similar anatomy, or reachable endoluminally through such passageways. Such devices typically include a flexible elongate shaft, so as to traverse tortuous luminal anatomy, and an energy delivery element mounted thereon to deliver such energy to remote or enclosed locations such as body lumens. Such devices have been developed to treat, for example, passageways of the lungs or blood vessels of the vasculature, or to treat various organs such as the heart, stomach, intestines, etc. In other instances, delivery is direct through open surgery or through a percutaneous approach. Different environments, such as airways versus blood filled environments, and differing diseases, such as those affecting the surface tissues versus those affecting deeper layers or tissues, lead to varying objectives for these devices.

The PEF energy disrupts the integrity of the target tissue cells which initiates a cascade of biochemical processes which induce different forms of cell death including necrosis, apoptosis, aponeurosis, necroptosis, and/or pyroptosis. Since PEF therapies are not dependent on thermal processes, cells are killed within a volume of tissue in-vivo without altering the stromal proteins and extracellular matrix within that volume and thus facilitating the preserved function of those critical and sensitive anatomic structures, such as the major vasculature, luminal systems such as the common bile duct, and tissue structures such as the pleura. Thus. PEF therapies offer a superior safety profile relative to other focally ablative modalities. As a consequence of those characteristics. PEF is being applied in a variety of different disease states with increasing regularity, including cancer, heart disease, and lung disease.

Variations exist between clinical PEF systems, including differences in waveform parameters and delivery polarity (i.e., bipolar or monopolar). In bipolar electrode configurations, energy is delivered between effector devices placed within, or adjacent to, the targeted environment. Conversely, monopolar systems use a single end-effector to deliver energy to the targeted location with a remote dispersive electrode serving as the electrical return. The dispersive electrode is of sufficient surface area to distribute the PEF energy broadly enough that no treatment effects are encountered at its remote location.

An important consideration regarding clinical use of PEF energy is its potential to stimulate cardiac tissue and interfere with the normal cardiac cycle. This is particularly the case with monopolar delivery arrangements which penetrate the body more deeply than delivery using bipolar electrode arrangements. In addition, several other variables impact the arrhythmogenic potential of any given PEF therapy including the location, extent, and timing of this stimulation. Many PEF technologies reduce arrhythmogenesis by synchronizing PEF delivery to the cardiac refractory period (i.e., ST interval). However, such synchronization involves additional equipment, such as an external cardiac monitor and a mechanism for acquiring an electrocardiogram (ECG). In some instances, the cardiac monitor is used to continuously acquire an ECG signal via external electrodes that are positioned on the patient's chest. The PEF generator analyzes one or more cardiac cycles and identifies the beginning of a time period during which it is safe to apply energy to the patient, thus providing the ability to synchronize energy delivery with the cardiac cycle. In some embodiments, this time period is within milliseconds of the R wave (of the ECG QRS complex) to avoid induction of an arrhythmia, which could occur if the energy pulse is delivered on a T wave. Such synchronization also complicates the delivery of the energy in that energy delivery is limited to periods allowed by synchronization.

Improved delivery of PEF energy is desired for efficiency, simplicity, reduced cost and improved patient outcomes, to name a few. At least some of these objectives will be met by the systems, devices and methods described herein.

### SUMMARY OF THE INVENTION

Described herein are embodiments of apparatuses, systems and methods for treating target tissue in the body. Likewise, the invention relates to the following numbered clauses:
1. A system for delivering energy to a target tissue within a torso of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein the energy has a waveform comprising a plurality of pulses each having a voltage of at least 1000V and each is below a threshold for induction of arrhythmia.
2. A system as in clause 1, wherein the plurality of pulses comprises biphasic pulses.
3. A system as in any of the above clauses, wherein the voltage is in a range of 1000 to 10,000V.
4. A system as in any of the above clauses, wherein the pulses are spaced so that at least one of the pulses is delivered to the target tissue during at least a portion of a T-wave of the cardiac cycle.
5. A system as in clause 4, wherein the pulses are spaced so that at least one of the pulses is delivered to the target tissue during a middle and/or terminal phase of the T-wave of the cardiac cycle.
6. A system as in any of clauses 1-3, wherein the pulses are spaced so that at least one of the pulses is delivered to the target tissue during atrial contraction of the cardiac cycle.
7. A system as in any of clauses 1-3, wherein the pulses are spaced so that at least one of the pulses is delivered to the target tissue during a maximum contraction peak of atrial systole or ventricular systole of the cardiac cycle.
8. A system as in any of the above clauses, wherein the plurality of pulses comprises at least one packet of pulses.
9. A system as in clause 8, wherein the at least one packet of pulses comprises at least six packets of pulses, wherein each packet includes 40 biphasic pulses with inter-pulse delays of 1000 microseconds.
10. A system as in any of the above clauses, wherein each pulse has a pulse duration of less than or equal to 10 μs.
11. A system as in clause 10, wherein each pulse has a pulse duration in a range of 0.5 us to 5 μs.
12. A system as in clause 10, wherein the pulses each have a voltage in a range of 1000-10,000V.
13. A system as in clause 12, wherein the pulses each have a voltage in a range of 1000-5000V.
14. A system as in any of clauses 1-9, wherein the pulses each have a voltage in a range of 1000-3000V and wherein each pulse has a pulse duration in a range of less than or equal to 25 μs.
15. A system as in any of clauses 1-9, wherein the pulses each have a voltage in a range of 1000-1500V and wherein each pulse has a pulse duration in a range of less than or equal to 50 US.
16. A system as in any of the above clauses, wherein the dose has a delivery time of at least the cardiac cycle.
17. A system as in any of the above clauses, wherein the target tissue resides within a lung of the patient.
18. A system as in any of clauses 1-16, wherein the target tissue comprises resides within a gastrointestinal system, urinary or reproductive system of the patient.
19. A system as in any of clauses 1-16, wherein the energy is delivered at a constant delivery rate and wherein the target tissue comprises cardiac tissue, the system further comprising a robotic apparatus programmed to drag the energy delivery body along the cardiac tissue during energy delivery.
20. A system as in any of claims 1-18, wherein the target tissue comprises a tumor and the energy delivery body comprises a probe.
21. A system as in any of the above claims, wherein the energy is delivered at a constant delivery rate.
22. A system as in any of the above claims, further comprising a robotic apparatus for manipulating the energy delivery body within the patient.
23. A system as in any of the above clauses, wherein energy delivery is synced with respiration of the patient without cardiac synchronization.
24. A system as in clause 23, wherein the energy delivery is actuated when the lung is not in motion.
25. A system as in any of clauses 1-22, wherein energy delivery is disassociated from syncing to the cardiac cycle and is actuated by feedback control.
26. A system as in clause 25, wherein the feedback control comprises temperature monitoring, impedance monitoring, pH monitoring, contact detection and/or contact force.
27. A system as in clause 25, further comprising one or more sensors configured to detect an agent within the body and wherein energy delivery is actuated by feedback from the one or more sensors.
28. A system as in clause 27, wherein the agent comprises a drug, molecule, gene, chemotherapeutic agent.
29. A system as in any of clauses 27-28, wherein the energy delivery body comprises at least one tine extending from a catheter and wherein the system further comprises one or more impedance sensors configured to measure impedance between one of the at least one tine and another of the at least one tine or a portion of the catheter.
30. A system as in clause 29, wherein the one or more impedance sensors comprise a pH sensor, a fiber optic sensor, or a bipolar impedance sensor.
31. A system for delivering energy to a target tissue within a torso of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein the dose is comprised of a plurality of pulses wherein at least one of the pulses is arranged within the dose so as to be received by the target tissue during a T-wave of the cardiac cycle and wherein the dose does not induce arrhythmia.
32. A system as in clause 31, wherein the plurality of pulses comprises biphasic pulses.
33. A system as in any of clauses 31-32, wherein the voltage is in a range of 1000 to 10,000V.
34. A system as in clause 31, wherein the pulses are spaced so that at least one of the pulses is delivered to the target tissue during a middle and/or terminal phase of the T-wave of the cardiac cycle.
35. A system as in any of clauses 31-34, wherein the plurality of pulses comprises at least one packet of pulses.
36. A system as in clause 35, wherein the at least one packet of pulses comprises at least six packets of pulses, wherein each packet includes 40 biphasic pulses with inter-pulse delays of 1000 microseconds.
37. A system as in any of clauses 31-36, wherein each pulse has a pulse duration of less than or equal to 10 μs.
38. A system as in clause 37, wherein each pulse has a pulse duration in a range of 0.5 μs to 5 μs.
39. A system as in clause 37, wherein the pulses each have a voltage in a range of 1000-10,000V.
40. A system as in clause 39, wherein the pulses each have a voltage in a range of 1000-5000V.
41. A system as in any of clauses 31-36, wherein the pulses each have a voltage in a range of 1000-3000V and wherein each pulse has a pulse duration in a range of less than or equal to 25 μs.
42. A system as in any of clauses 31-36, wherein the pulses each have a voltage in a range of 1000-1500V and wherein each pulse has a pulse duration in a range of less than or equal to 50 μs.
43. A system as in any of clauses 31-42, wherein the dose has a delivery time of at least the cardiac cycle.
44. A system as in any of clauses 31-43, wherein the target tissue resides within a lung of the patient.
45. A system as in any of clauses 31-43, wherein the target tissue comprises resides within a gastrointestinal system, urinary or reproductive system of the patient.
46. A system as in any of clauses 31-45, wherein the energy is delivered at a constant delivery rate and wherein the target tissue comprises cardiac tissue, the system further comprising a robotic apparatus programmed to drag the energy delivery body along the cardiac tissue during energy delivery.
47. A system as in any of clauses 31-46, wherein the target tissue comprises a tumor and the energy delivery body comprises a probe.
48. A system as in any of clauses 31-47, wherein the energy is delivered at a constant delivery rate.
49. A system as in any of clauses 31-48, further comprising a robotic apparatus for manipulating the energy delivery body within the patient.
50. A system as in any of clauses 31-49, wherein energy delivery is synced with respiration of the patient without cardiac synchronization.
51. A system as in clause 50, wherein the energy delivery is actuated when the lung is not in motion.
52. A system for delivering energy to a target tissue within a torso of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein the dose is comprised of a plurality of pulses and wherein the dose has a delivery time of at least the cardiac cycle.
53. A system as in clause 52, wherein the plurality of pulses comprises at least one packet of pulses.
54. A system as in clause 53, wherein the at least one packet of pulses comprises at least six packets of pulses, wherein each packet includes 40 biphasic pulses with inter-pulse delays of 1000 microseconds.
55. A system as in any of clauses 52-54, wherein each pulse has a pulse duration of less than or equal to 10 μs.
56. A system as in clause 55, wherein each pulse has a pulse duration in a range of 0.5 μs to 5 μs.
57. A system as in clause 55, wherein the pulses each have a voltage in a range of 1000-10,000V.
58. A system as in clause 57, wherein the pulses each have a voltage in a range of 1000-5000V.
59. A system as in any of clauses 52-58, wherein the pulses each have a voltage in a range of 1000-3000V and wherein each pulse has a pulse duration in a range of less than or equal to 25 μs.
60. A system as in any of clauses 52-58, wherein the pulses each have a voltage in a range of 1000-1500V and wherein each pulse has a pulse duration in a range of less than or equal to 50 μs.
61. A system as in any of clauses 52-60, wherein the dose has a delivery time of at least the cardiac cycle.
62. A system as in any of clauses 52-61, wherein the target tissue resides within a lung of the patient.
63. A system as in any of clauses 52-61, wherein the target tissue comprises resides within a gastrointestinal system, urinary or reproductive system of the patient.
64. A system as in any of claims 52-61 wherein the energy is delivered at a constant delivery rate and wherein the target tissue comprises cardiac tissue, the system further comprising a robotic apparatus programmed to drag the energy delivery body along the cardiac tissue during energy delivery.
65. A system as in any of clauses 52-63, wherein the target tissue comprises a tumor and the energy delivery body comprises a probe.
66. A system as in any of clauses 52-65, wherein the energy is delivered at a constant delivery rate.
67. A system as in any of clauses 52-66, further comprising a robotic apparatus for manipulating the energy delivery body within the patient.
68. A system for delivering energy to a target tissue within a torso of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein energy delivery is disassociated from syncing to the cardiac cycle and is actuated by feedback control.
69. A system as in clause 68, wherein the feedback control comprises temperature monitoring, impedance monitoring, pH monitoring, contact detection and/or contact force.
70. A system as in clause 68, further comprising one or more sensors configured to detect an agent within the body and wherein energy delivery is actuated by feedback from the one or more sensors.
71. A system as in clause 70, wherein the agent comprises a drug, molecule, gene, or chemotherapeutic agent.
72. A system as in any of clauses 70-71, wherein the energy delivery body comprises at least one tine extending from a catheter and wherein the system further comprises one or more impedance sensors configured to measure impedance between one of the at least one tine and another of the at least one tine or a portion of the catheter.
73. A system as in clause 72, wherein the one or more impedance sensors comprise a pH sensor, a fiber optic sensor, or a bipolar impedance sensor.
74. A system for delivering energy to treat a target tissue within a patient that is close enough to their heart to cause arrhythmia comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue in a manner that is not synced with a cardiac cycle of the heart and does not induce arrhythmia.
75. A system as in clause 74, wherein the plurality of pulses comprises biphasic pulses.
76. A system as in any of clauses 74-75, wherein the voltage is in a range of 1000 to 10,000V.
77. A system as in any of clauses 74-76, wherein the pulses are spaced so that at least one of the pulses is delivered to the target tissue during at least a portion of a T-wave of the cardiac cycle.
78. A system as in clause 77, wherein the pulses are spaced so that at least one of the pulses is delivered to the target tissue during a middle and/or terminal phase of the T-wave of the cardiac cycle.
79. A system as in any of clauses 74-76, wherein the pulses are spaced so that at least one of the pulses is delivered to the target tissue during atrial contraction of the cardiac cycle.
80. A system as in any of clauses 74-76, wherein the pulses are spaced so that at least one of the pulses is delivered to the target tissue during a maximum contraction peak of atrial systole or ventricular systole of the cardiac cycle.
81. A system as in any of clauses 74-80, wherein the plurality of pulses comprises at least one packet of pulses.
82. A system as in clause 81, wherein the at least one packet of pulses comprises at least six packets of pulses, wherein each packet includes 40 biphasic pulses with inter-pulse delays of 1000 microseconds.
83. A system as in any of clauses 74-82, wherein each pulse has a pulse duration of less than or equal to 10 μs.
84. A system as in clause 83, wherein each pulse has a pulse duration in a range of 0.5 μs to 5 μs.
85. A system as in clause 83, wherein the pulses each have a voltage in a range of 1000-10,000V.
86. A system as in clause 85, wherein the pulses each have a voltage in a range of 1000-5000V.
87. A system as in any of clauses 74-82, wherein the pulses each have a voltage in a range of 1000-3000V and wherein each pulse has a pulse duration in a range of less than or equal to 25 US.
88. A system as in any of clauses 74-82, wherein the pulses each have a voltage in a range of 1000-1500V and wherein each pulse has a pulse duration in a range of less than or equal to 50 μs.
89. A system as in any of clauses 74-88, wherein the dose has a delivery time of at least the cardiac cycle.
90. A system as in any of claims 74-89, wherein the target tissue resides within a lung of the patient.
91. A system as in any of clauses 74-89, wherein the target tissue comprises resides within a gastrointestinal system, urinary or reproductive system of the patient.
92. A system as in any of clauses 74-89, wherein the energy is delivered at a constant delivery rate and wherein the target tissue comprises cardiac tissue, the system further comprising a robotic apparatus programmed to drag the energy delivery body along the cardiac tissue during energy delivery.
93. A system as in any of clauses 74-91, wherein the target tissue comprises a tumor and the energy delivery body comprises a probe.
94. A system as in any of the clauses 74-93, wherein the energy is delivered at a constant delivery rate.
95. A system as in any of the clauses 74-94, further comprising a robotic apparatus for manipulating the energy delivery body within the patient.
96. A system as in any of clauses 74-95, wherein energy delivery is synced with respiration of the patient without cardiac synchronization.
97. A system as in clause 96, wherein the energy delivery is actuated when the lung is not in motion.
98. A system as in any of clauses 74-95, wherein energy delivery is actuated by feedback control.
99. A system as in clause 98, wherein the feedback control comprises temperature monitoring, impedance monitoring, pH monitoring, contact detection and/or contact force.
100. A system as in clause 98, further comprising one or more sensors configured to detect an agent within the body and wherein energy delivery is actuated by feedback from the one or more sensors.
101. A system as in clause 100, wherein the agent comprises a drug, molecule, gene, chemotherapeutic agent.
102. A system as in any of clauses 100-101, wherein the energy delivery body comprises at least one tine extending from a catheter and wherein the system further comprises one or more impedance sensors configured to measure impedance between one of the at least one tine and another of the at least one tine or a portion of the catheter.
103. A system as in clause 102, wherein the one or more impedance sensors comprise a pH sensor, a fiber optic sensor, or a bipolar impedance sensor.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an embodiment of a tissue modification system.

FIGS. 2A-2B illustrate another embodiment of a tissue modification system.

FIG. 3 illustrates an embodiment of a waveform of a signal prescribed by an energy delivery algorithm.

FIG. 4 provides a table illustrating various example effects of parameter changes.

FIG. 5A illustrates simulation tissue electrical conductivity as a function of electric field exposure.

FIG. 5B illustrates simulated electric current and impedance as a function of simulation applied voltages for 500-5000V.

FIGS. 6A-6C illustrate A) Reference data. B) Power law curve fitting for the “Stimulation (coil)” curve from (A). C) Power law curve fitting for the “Induction” curve from (A): Note: “Stimulation (coil)” was used for stimulation since it reflects the same electrode relationship that was used to generate the “Induction” data.

FIG. 7 illustrates an embodiment of a monopolar system used in the evaluation of the specialized PEF energy.

FIGS. 8A-8D illustrate numerical simulation representative results: (FIG. 8A) Numerical simulation geometry and (FIGS. 8B-8D) Cross-sectional view of electrical conductivity, electric field, and voltage distributions into simulated lung parenchymal tissue. Grid=5 mm.

FIGS. 9A-9B illustrate PEF cardiac effect sensitivity in relation to basic treatment.

FIGS. 10A-10D illustrate a fluoroscopic image of basket deployment with CT images of porcine chest.

FIGS. 11A-11D illustrate examples of cardiac effects from asynchronous PEF delivery: there may be no change (FIG. 11A), an artifactual change in the immediate heartbeat ECG due to signal interference (FIG. 11B), a PAC without ventricular conduction that briefly delays the subsequent heartbeat (FIG. 11C), or a PAC that conducts through to the ventricle resulting in an early heartbeat (FIG. 11D).

### DETAILED DESCRIPTION

Devices, systems and methods are provided for treating tissues of the body, with pulsed electric field (PEF) energy. PEF energy is typically characterized as high voltage pulsed energy that is configured to be delivered in one or more doses. Each energy dose delivered to the target tissue is configured to maintain the temperature at or in the target tissue below a threshold for thermal ablation. Instead of inducing thermal damage from thermal ablation, described as extracellular protein coagulation, the effects are considered non-thermal wherein such energy modifies or destroys cells within the tissue but preserves the underlying protein extracellular matrix of tissues that provides the interstitial architectural structure and structure-related functions of the tissue. In some instances, this allows regeneration of tissue, such as by repopulation of the extracellular matrices. In addition, nearby sensitive tissues are spared injury. It may be appreciated that the doses may be titrated or moderated over time so as to further reduce or eliminate thermal buildup during the treatment procedure. It may be appreciated that in some embodiments energy delivery is actuated by a variety of mechanisms, such as with the use of an actuator on the device or a foot switch operatively connected to a generator. Such actuation typically provides a single energy dose or activation.

Target tissue cells may be treated in any location throughout the body, including cells of the gastrointestinal or digestive system (e.g. mouth, glands, esophagus, stomach, duodenum, jejunum, ileum, intestines, colon, rectum, liver, gall bladder, pancreas, anal canal, etc.), cells of the respiratory system (e.g. nasal cavity, pharynx larynx, trachea, bronchi, lungs, etc.), cells of the urinary system (e.g. kidneys, ureter, bladder, urethra, etc.), cells of the reproductive system (e.g. reproductive organs, ovaries, fallopian tubes, uterus, cervix, vagina, testes, epididymis, vas deferens, seminal vesicles, prostate, glands, penis, scrotum, etc.), cells of the endocrine system (e.g. pituitary gland, pineal gland, thyroid gland, parathyroid gland, adrenal gland), cells of the circulatory system (e.g. heart, arteries, veins, etc.), cells of the lymphatic system (e.g. lymph node, bone marrow, thymus, spleen, etc.), cells of the nervous system (e.g. brain, spinal cord, nerves, ganglia, etc.), cells of the eye (e.g. retina, macula, Layer of Rods and Concs, retinal pigment epithelium optic nerve, choroid, sclera, etc.), cells of the muscular system (e.g. myocytes, etc.), and cells of the skin (e.g. epidermis, dermis, hypodermis, etc.), to name a few.

Conditions treated include arrhythmias, particularly atrial fibrillation. When treating atrial fibrillation, lesion rings, such as around the outside ostium of a pulmonary vein, are typically created by the delivery of PEF energy with either focal catheters or one-shot catheters. In addition, the focal catheters can be used to create many other types of lesions, particularly lines along various surfaces of cardiac tissue. In one embodiment, a cavo-tricuspid isthmus line is created for the treatment of typical atrial flutter in the right atrium. In another embodiment, roof lines and/or floor lines are created for a box lesion along the posterior wall of the left atrium for patients with atrial fibrillation, particularly for persistent atrial fibrillation. In another embodiment, a mitral isthmus line is created along the anterior or lateral wall of the left atrium for atypical atrial flutter. In yet another embodiment, ventricular lines are created connecting two inexcitable boundaries that are critical to the initiation or maintenance of a reentrant ventricular arrhythmia, typically in patients with ventricular tachycardia resulting from ischemic heart disease.

Other conditions treated include pulmonary disorders, such as chronic obstructive pulmonary disease, chronic bronchitis, mucus hypersecretion, asthma and cystic fibrosis, to name a few. Further, a variety of

Still other conditions comprise a coagulation disorder, such as hemophilia (e.g., hemophilia A or hemophilia B), von Willebrand's disease, factor XI deficiency, a fibrinogen disorder, or a vitamin K deficiency. The coagulation disorder may be characterized by a mutation in a gene encoding for fibrinogen, prothrombin, factor V, factor VII, factor VIII, factor X, factor XI, factor XIII, or an enzyme involved in posttranslational modifications thereof, or an enzyme involved in vitamin K metabolism. In some embodiments, the coagulation disorder is characterized by a mutation in FGA, FGB, FGG, F2, F5, F7, F10, F11, F13A, F13B, LMANI, MCFD2, GGCX, or VKORC1.

In some embodiments, the disorder comprises a neurological disorder, e.g., a neurodegenerative disease. In some embodiments, the neurodegenerative disease comprises Alzheimer's disease. Parkinson's disease, or multiple sclerosis. In some embodiments, the neurodegenerative disease comprises an autoimmune disease of the central nervous system (CNS), such as multiple sclerosis, encephalomyelitis, a paraneoplastic syndrome, autoimmune inner car disease, or opsoclonus myoclonus syndrome. The neurological disorder may be a cerebral infarction, spinal cord injury, central nervous system disorder, a neuropsychiatric disorder, or a channelopathy (e.g., epilepsy or migraine). The neurological disorder may be an anxiety disorder, a mood disorder, a childhood disorder, a cognitive disorder, schizophrenia, a substance related disorders, or an eating disorder. In some embodiments, the neurological disorder is a symptom of a cerebral infarction, stroke, traumatic brain injury, or spinal cord injury.

In some embodiments, the disorder comprises a lysosomal storage disorder, such as Tay-Sachs disease, Gaucher disease, Fabry disease, Pompe disease, Niemann-Pick disease, or mucopolysaccharidosis (MPS).

In some embodiments, the disorder comprises a cardiovascular disorder, such as a degenerative heart disease, a coronary artery disease, an ischemia, angina pectoris, an acute coronary syndrome, a peripheral vascular disease, a peripheral arterial disease, a cerebrovascular disease, or atherosclerosis. The cardiovascular disorder may be a degenerative heart disease selected from the group consisting of an ischemic cardiomyopathy, a conduction disease, and a congenital defect.

In some embodiments, the disorder comprises an immune disorder, e.g., an autoimmune disorder. The autoimmune disorder may be type 1 diabetes, multiple sclerosis, rheumatoid arthritis, lupus, encephalomyelitis, a paraneoplastic syndrome, autoimmune inner ear disease, or opsoclonus myoclonus syndrome, autoimmune hepatitis, uveitis, autoimmune retinopathy, neuromyelitis optica, psoriatic arthritis, psoriasis, myasthenia gravis, chronic Lyme disease, celiac disease, chronic inflammatory demyelinating polyneuropathy, peripheral neuropathy, fibromyalgia. Hashimoto's thyroiditis, ulcerative colitis, or Kawasaki disease.

In some embodiments, the disorder comprises a liver disease, such as hepatitis, Alagille syndrome, biliary atresia, liver cancer, cirrhosis, a cystic disease, Caroli's syndrome, congenital hepatic fibrosis, fatty liver, galactosemia, primary sclerosing cholangitis, tyrosinemia, glycogen storage disease, Wilson's disease, or an endocrine deficiency. The liver disease may be a liver cancer such as a hepatocellular hyperplasia, a hepatocellular adenoma, a focal nodular hyperplasia, or a hepatocellular carcinoma.

In some embodiments, the disorder comprises a tumor or cancer, such as a blood cancer (e.g., acute lymphoblastic leukemia, acute myeloblastic leukemia, chronic myelogenous leukemia, Hodgkin's disease, multiple myeloma, and non-Hodgkin's lymphoma) or a solid tissue cancer (e.g., lung cancer, liver cancer, kidney cancer, a breast cancer, a gastric cancer, an esophageal cancer, a stomach cancer, an intestinal cancer, a colorectal cancer, a bladder cancer, a prostate cancer, a head and neck cancer, a skin cancer, or a brain cancer, to name a few).

In some embodiments, the disorder comprises a recessively inherited disorder. In some embodiments, the disorder is a Mendelian-inherited disorder.

In some embodiments, the disorder comprises an ocular disorder that is a retinal dystrophy (e.g., a Mendelian-heritable retinal dystrophy). The retinal dystrophy may be comprised of leber's congenital amaurosis (LCA), Stargardt Disease, pseudoxanthoma elasticum, rod cone dystrophy, exudative vitreoretinopathy. Joubert Syndrome, CSNB-1C, age-related macular degeneration, retinitis pigmentosa, stickler syndrome, microcephaly and choriorretinopathy, retinitis pigmentosa, CSNB 2, Usher syndrome, or Wagner syndrome.

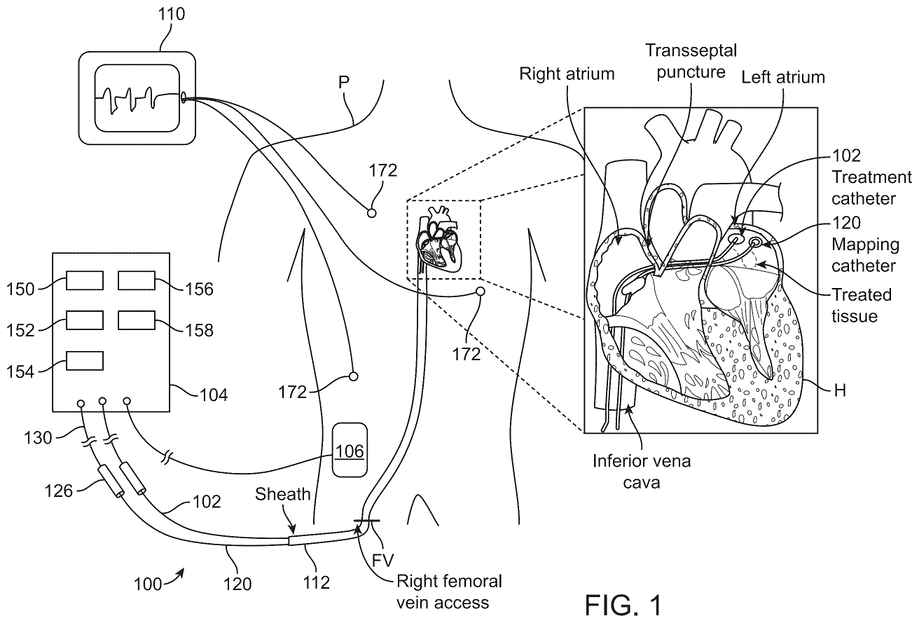

FIG. 1 illustrates an example tissue modification system 100 that is used to deliver PEF energy to target tissue in the heart. In this embodiment, the tissue modification system 100 includes a specialized catheter 102, a high voltage waveform generator 104 and at least one distinct energy delivery algorithm 152. In this embodiment the generator 104 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery, however any other suitable energy storage element may be used. In addition, one or more communication ports are included. In this embodiment, the treatment catheter 102 is designed to be monopolar, wherein the distal end of the catheter 102 has at least one delivery energy delivery body (e.g. electrode) and a return electrode 106 is positioned upon the skin outside the body, typically on the thigh, lower back or back. In this embodiment, the heart H is accessed via the right femoral vein FV by a suitable access procedure, such as the Seldinger technique. Typically, an introducer sheath 112 is inserted into the femoral vein FV which acts as a conduit through which various catheters and/or tools may be advanced, including the treatment catheter 102. The distal end of the catheter 102 is advanced through the inferior vena cava, through the right atrium, through a transseptal puncture, and into the left atrium so as to access the entrances to the pulmonary veins. In this embodiment, the catheter 102 is used to perform cardiac mapping which refers to the process of identifying the temporal and spatial distributions of myocardial electrical potentials during a particular heart rhythm. Cardiac mapping during an aberrant heart rhythm aims at elucidation of the mechanisms of the heart rhythm, description of the propagation of activation from its initiation to its completion within a region of interest, and identification of the site of origin or a critical site of conduction to serve as a target for treatment. Once the desired treatment locations are identified, the catheter 102 is utilized to deliver the treatment energy.

Additional example embodiments of energy delivery catheters 102 configured to provide focal therapy to various parts of the body are provided in international patent application number PCT/US2018/067504 titled “OPTIMIZATION OF ENERGY DELIVERY FOR VARIOUS APPLICATIONS” which claims priority to Provisional Patent Application No. 62/610,430 filed Dec. 26, 2017 and U.S. Provisional Patent Application No. 62/693,622 filed Jul. 3, 2018, all of which are incorporated herein by reference for all purposes. In another example, in some embodiments, the energy delivery catheter 102 may have a variety of end effectors such as according to Provisional Patent Application 63/159,331 titled “DEVICES FOR THE DELIVERY OF PULSED ELECTRIC FIELDS IN THE TREATMENT OF CARDIAC TISSUE” filed Mar. 10, 2021, all of which are incorporated herein by reference for all purposes. Likewise, treatment energy may be delivered with a variety of catheter designs, optionally with the use of a variety of accessories, such as according to international patent application number PCT/US2020/066205 titled “TREATMENT OF CARDIAC TISSUE WITH PULSED ELECTRIC FIELDS”, filed Dec. 18, 2020 which claims priority to Provisional Patent Application No. 62/949,633 filed Dec. 18, 2019, Provisional Patent Application No. 63/000,275 filed Mar. 26, 2020 and Provisional Patent Application No. 63/083,644 filed Sep. 25, 2020, all of which are incorporated by reference for all purposes.

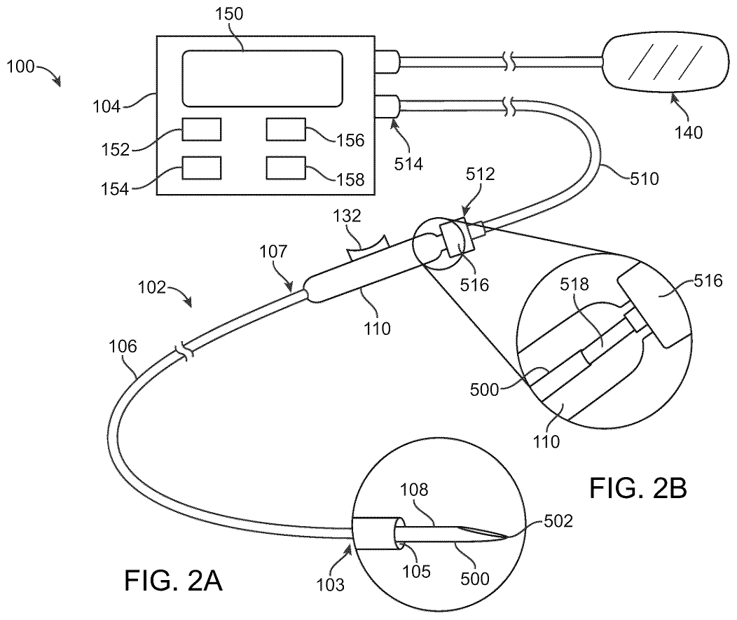

FIGS. 2A-2B, illustrate another embodiment of a tissue modification system 100 comprising an energy delivery catheter 102 connectable with a generator 104. As shown, the catheter 102 comprises a shaft 106 having a distal end 103, a proximal end 107 and at least one lumen 105 extending at least partially therethrough. Likewise, the catheter 102 also includes at least one energy delivery body 108. In this embodiment, an energy delivery body 108 has the form of a probe 700 that is disposed within the lumen 105 of the shaft 106. The probe 700 has a probe tip 702 that is advanceable through the lumen 105 and extendable from the distal end 103 of the shaft 106 (expanded in FIG. 2A to show detail). In this embodiment, the tip 702 has a pointed shape configured to penetrate tissue, such as to resemble a needle. Thus, in this embodiment, the probe tip 702 is utilized to penetrate the lumen wall W and surrounding tissue so that it may be inserted into the target tissue external to the body lumen. Thus, the probe 700 has sufficient flexibility to be endoluminally delivered yet has sufficient column strength to penetrate the lumen wall W and target tissue. In some embodiments, the catheter 102 has markings to indicate to the user the distance that the probe tip 702 has been advanced so as to ensure desired placement.

In some embodiments, the probe extends from the distal end 103 of the shaft 106 approximately less than 0.5 cm, 0.5 cm, 1 cm, 2 cm, 3 cm, 4 cm, 5 cm, 6 cm, 7 cm, 8 cm or more than 8 cm. In some embodiments, the probe extends 1-3 cm or 2-3 cm from the distal end of the shaft 106. In some embodiments, the probe is 18 gauge, 19 gauge, 20 gauge, 21 gauge, 22 gauge, 23 gauge, 24 gauge, or 25 gauge. In some embodiments, the probe 700 is comprised of a conductive material so as to serve as an electrode. Thus, the electrode would have the size of the exposed probe. Example materials include stainless steel, nitinol, cobalt-chromium alloy, copper, and gold. In some embodiments, the exposed probe conductive material is coated with a different material, with examples including platinum-iridium, gold, platinum black, palladium, or other materials. The conductive material, or the conductive material coating may be designed so as to reduce the biological interactions of the tissue, reduce the production of electrochemical effects from the PEF treatment, or more efficiently distribute the PEF energy into the tissue, among other purposes. The materials may be smooth, electropolished, sandblasted at various grits, or treated with other mechanical or chemical preparations to alter the roughness of the surface, which may be done to reduce biological interactions of the tissue, facilitate easier deployment and retraction of the electrode, reduce the production of electrochemical effects from the PEF treatment, or more efficiently distribute the PEF energy into the tissue, among other purposes. Thus, in these embodiments, the PEF energy is transmittable through the probe 700 to the probe tip 702. Consequently, the shaft 106 is comprised of an insulating material or is covered by an insulating sheath. Example insulating materials include polyimide, silicone, polytetrafluoroethylene, and polyether block amide. The insulating material may be consistent or varied along the length of the shaft 106 or sheath. Likewise, in either case, the insulating material typically comprises complete electrical insulation. However, in some embodiments, the insulating material allows for some leakage current to penetrate.

When the probe 700 is energized, the insulting shaft 106 protects the surrounding tissue from the treatment energy and directs the energy to the probe tip 702 (and any exposed portion of the probe 700) which is able to deliver treatment energy to surrounding tissue. Thus, the tip 702 acts as a delivery electrode and its size can be selected based on the amount of exposed probe 700. Larger electrodes can be formed by exposing a greater amount of the probe 700 and smaller electrodes can be formed by exposing less. In some embodiments, the exposed tip 702 (measured from its distal end to the distal edge of the insulating shaft) during energy delivery has a length of 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, greater than 3 cm, up to 8 cm, less than or equal to 0.1 cm, less than or equal to 0.3 cm, less than or equal to 0.5 cm, less than or equal to 1 cm, 0.2-0.3 cm, 0.1-0.5 cm, 0.1-1 cm, and all ranges and subranges therebetween. In addition to changing the size of the electrode, the tip 702 is retractable into the shaft 106 to allow for atraumatic endoscopic delivery and is then advanceable as desired to reach the target tissue. In this embodiment, advancement and retraction are controlled by an actuator 732 (e.g. knob, button, lever, slide or other mechanism) on a handle 110 attached to the proximal end 107 of the shaft 106. It may be appreciated that the shaft 106 itself may be advanced toward the target tissue, with or without advancing the probe from the distal end 103 of the shaft 106. In some embodiments, the distal end of the shaft 106 is advanced up to 20 cm into the tissue, such as from an external surface of a luminal structure or from an external surface of the body of the patient.

The handle 110 is connected to the generator 104 with the use of a specialized energy plug 510. The energy plug 510 has a first end 512 that connects to the handle 110 and a second end 514 the connects to the generator 104. The connection of the first end 512 with the handle 110 is expanded for detail in FIG. 2B. In this embodiment, the first end 712 has an adapter 716 that includes a connection wire 718 extending therefrom. The connection wire 718 is insertable into the proximal end of the probe 700 within the handle 110. This allows the energy to be transferred from the generator 104, through the connection wire 718 to the probe 700. Thus, the probe 700 is able to be electrified throughout its length, however only the exposed tip 702 delivers energy to the tissue due to the presence of the insulated shaft 106.

In this embodiment, the generator 104 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery, however any other suitable energy storage element may be used. In addition, one or more communication ports are included.

In some embodiments, the generator 104 includes three sub-systems: 1) a high-energy storage system, 2) a high-voltage, medium-frequency switching amplifier, and 3) the system controller, firmware, and user interface. In some embodiments, the system controller includes a synchronization trigger monitor that allows for synchronizing the pulsed energy output to a desired trigger. The generator takes in alternating current (AC) mains to power multiple direct current (DC) power supplies. The generator's controller can cause the DC power supplies to charge a high-energy capacitor storage bank before energy delivery is initiated. At the initiation of therapeutic energy delivery, the generator's controller, high-energy storage banks and a bi-phasic pulse amplifier can operate simultaneously to create a high-voltage, medium frequency output.

It will be appreciated that a multitude of generator electrical architectures may be employed to execute the energy delivery algorithms. In particular, in some embodiments, advanced switching systems are used which are capable of directing the pulsed electric field circuit to the energy delivering electrodes separately from the same energy storage and high voltage delivery system. Further, generators employed in advanced energy delivery algorithms employing rapidly varying pulse parameters (e.g., voltage, frequency, etc.) or multiple energy delivery electrodes may utilize modular energy storage and/or high voltage systems, facilitating highly customizable waveform and geographical pulse delivery paradigms. It should further be appreciated that the electrical architecture described herein above is for example only, and systems delivering pulsed electric fields may or may not include additional switching amplifier components.

The user interface 150 can include a touch screen and/or more traditional buttons to allow for the operator to enter patient data, select a treatment algorithm (e.g., energy delivery algorithm 152), initiate energy delivery, view records stored on the storage/retrieval unit 156, and/or otherwise communicate with the generator 104. The user interface 150 can include a voice-activated mechanism to enter patient data or may be able to communicate with additional equipment in the suite so that control of the generator 104 is through a secondary separate user interface.

In some embodiments, the user interface 150 is configured to receive operator-defined inputs. The operator-defined inputs can include a duration of energy delivery, one or more other timing aspects of the energy delivery pulse, power, and/or mode of operation, or a combination thereof. Example modes of operation can include (but are not limited to): system initiation and self-test, operator input, algorithm selection, pre-treatment system status and feedback, energy delivery, post energy delivery display or feedback, treatment data review and/or download, software update, or any combination or subcombination thereof.

In some embodiments, the processor 154, among other activities, modifies and/or switches between the energy-delivery algorithms, monitors the energy delivery and any sensor data, and reacts to monitored data via a feedback loop. In some embodiments, the processor 154 is configured to execute one or more algorithms for running a feedback control loop based on one or more measured system parameters (e.g., current), one or more measured tissue parameters (e.g., impedance), and/or a combination thereof.

The data storage/retrieval unit 156 stores data, such as related to the treatments delivered, and can optionally be downloaded by connecting a device (e.g., a laptop or thumb drive) to a communication port. In some embodiments, the device has local software used to direct the download of information, such as, for example, instructions stored on the data storage/retrieval unit 156 and executable by the processor 154. In some embodiments, the user interface 150 allows for the operator to select to download data to a device and/or system such as, but not limited to, a computer device, a tablet, a mobile device, a server, a workstation, a cloud computing apparatus/system, and/or the like. The communication ports, which can permit wired and/or wireless connectivity, can allow for data download, as just described but also for data upload such as uploading a custom algorithm or providing a software update.

The data storage/retrieval unit 156 can be, for example, a random access memory (RAM), a memory buffer, a hard drive, a database, an erasable programmable read-only memory (EPROM), an electrically erasable read-only memory (EEPROM), a read-only memory (ROM), flash memory, and/or so forth. The data storage/retrieval unit 156 can store instructions to cause the processor 154 to execute modules, processes and/or functions associated with the system 100.

Some embodiments the data storage/retrieval unit 156 comprises a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) can be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs). Compact Disc-Read Only Memories (CD-ROMs), and holographic devices; magneto-optical storage media such as optical disks; carrier wave signal processing modules; and hardware devices that are specially configured to store and execute program code, such as ASICs. Programmable Logic Devices (PLDs). Read-Only Memory (ROM) and Random-Access Memory (RAM) devices. Other embodiments described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using imperative programming languages (e.g., C. Fortran, etc.), functional programming languages (Haskell. Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

In some embodiments, the system 100 can be communicably coupled to a network, which can be any type of network such as, for example, a local area network (LAN), a wide area network (WAN), a virtual network, a telecommunications network, a data network, and/or the Internet, implemented as a wired network and/or a wireless network. In some embodiments, any or all communications can be secured using any suitable type and/or method of secure communication (e.g., secure sockets layer (SSL)) and/or encryption. In other embodiments, any or all communications can be unsecured.

As described herein, a variety of energy delivery algorithms 152 are programmable, or can be pre-programmed, into the generator 104, such as stored in memory or data storage/retrieval unit 156. Alternatively, energy delivery algorithms can be added into the data storage/retrieval unit to be executed by processor 154. The processor 154 can be, for example, a general-purpose processor, a field programmable gate array (FPGA), an application specific integrated circuit (ASIC), a digital signal processor (DSP), and/or the like. The processor 154 can be configured to run and/or execute application processes and/or other modules, processes and/or functions associated with the system 100, and/or a network associated with the system 100. As used herein the term “module” refers to any assembly and/or set of operatively-coupled electrical components that can include, for example, a memory, a processor, electrical traces, optical connectors, software (executing in hardware), and/or the like. For example, a module executed in the processor can be any combination of hardware-based module (e.g., a FPGA, an ASIC, a DSP) and/or software-based module (e.g., a module of computer code stored in memory and/or executed at the processor) capable of performing one or more specific functions associated with that module.

Each of these algorithms 152 may be executed by the processor 154. In some embodiments, the instrument 102 includes one or more sensors 160 that can be used to determine temperature, impedance, resistance, capacitance, conductivity, pH, optical properties (coherence, echogenicity, fluorescence), electrical or light permittivity, and/or conductance, to name a few. In some embodiments, one or more of the electrodes act as the one or more sensors. In other embodiments, the one or more sensors are separate from the electrodes. It may be appreciated that one or more sensors 160 may be disposed in a variety of locations, particularly depending on the parameter being sensed. For example, a sensor may be located along an energy delivery body 108, along an interior of the instrument, along the shaft 106, along an element that protrudes from the instrument, etc. Multiple sensors 160 may be present for sensing the same parameter at multiple sites, sensing different parameters at different sites, or sampling parameters at different sites to compile a single metric value measurement (e.g. average temperature, average voltage exposure, average conductivity, etc.). One or more sensors 160 may alternatively or additionally be located on a separate device. Sensor data can be used to plan the therapy, monitor the therapy and/or provide direct feedback via the processor 154, which can then alter the energy-delivery algorithm 152. For example, impedance measurements can be used to determine not only the initial dose to be applied but can also be used to determine the need for further treatment, or not.

It will be appreciated that the system 100 can include an automated treatment delivery algorithm that could dynamically respond and adjust and/or terminate treatment in response to inputs such as temperature, impedance at various voltages or AC frequencies, treatment duration or other timing aspects of the energy delivery pulse, treatment power and/or system status.

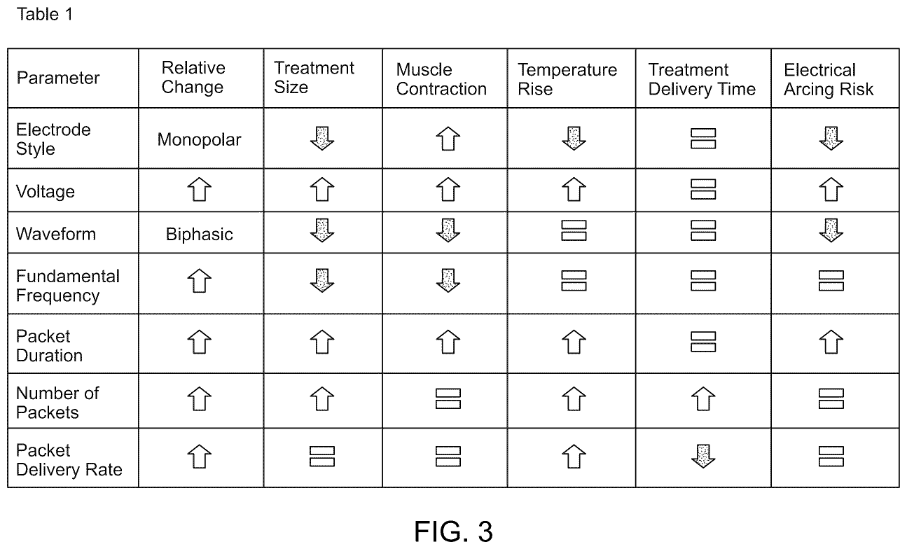

FIG. 3 illustrates an embodiment of a waveform 400 of a signal prescribed by an energy delivery algorithm 152. Here, two packets are shown, a first packet 402 and a second packet 404, wherein the packets 402, 404 are separated by a rest period or interpacket-delay 406. In this embodiment, each packet 402, 404 is comprised of a first biphasic cycle (comprising a first positive pulse peak 408 and a first negative pulse peak 410) and a second biphasic cycle (comprising a second positive pulse peak 408′ and a second negative pulse peak 410′). The first and second biphasic pulses are separated by dead time or inter-cycle delay 412 (i.e., a pause) between each pulse. In this embodiment, the biphasic pulses arc symmetric so that the set voltage 416 is the same for the positive and negative peaks. Here, the biphasic, symmetric waves are also square waves such that the magnitude and time of the positive voltage wave is approximately equal to the magnitude and time of the negative voltage wave.

It may be appreciated that manipulation of the various parameters (e.g. voltage, fundamental frequency, number of pulses per packet, number of packets, and various delays, etc.) have a variety of influences on the resultant lesion and on the body itself. In some instances, parameter changes can balance each other wherein the effect of change in one or more parameters can be balanced by a change in one or more different parameter values leading to the same or similar result. In other instances, parameter values can be tuned to create or generate different effects, such as different lesion characteristics (e.g. the presence, size and/or properties of the different zones) and/or different effects on the body. These effects on the body may be immediate, such as muscle stimulation, or delayed, such as the generation of a particular immune response.

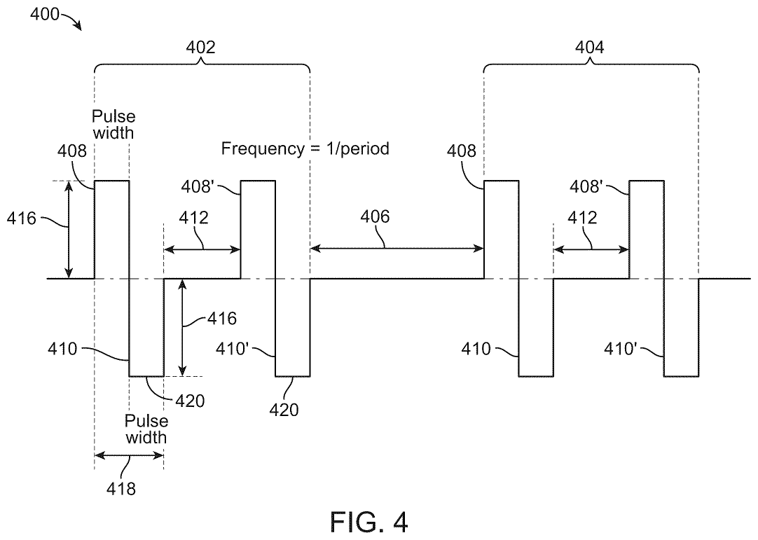

FIG. 4 provides a table (Table 1) illustrating various example effects of parameter changes. Typically, the electrode style is monopolar as opposed to bipolar. In monopolar arrangements, one or more delivery electrodes are positioned near the target tissue site and at least one remote return electrode is positioned against the patient's skin. By utilizing a monopolar electrode configuration, muscle contraction intensity increases. To counteract this effect, generally a biphasic waveform or waveform comprised of sufficiently short individual pulse durations comprising a packet with appropriate delays between the pulses, so as to offset the degree in muscle contraction. This waveform variety results in treatment effect decreases, but the decrease is more subtle than the muscle contraction reduction, thus resulting in still valid PEF therapeutic applications. Monopolar configurations also decrease the risk of electrical arcing compared to bipolar or multipolar configurations, wherein all effector electrodes are placed within a similar region that may permit electrical arcing between them. Typically, the waveform utilizes biphasic pulses as opposed to monophasic pulses. The use of biphasic pulses decreases the treatment size, decreases muscle contraction and also decreases arcing risk. Thus, the use of biphasic pulses counters the increase in muscle contraction due to the monopolar electrode style. Since both monopolar electrode style and biphasic pulses reduce treatment size, treatment size can be increased by changing other variables. For example, an increase in voltage, packet duration and number of packets increases the treatment size. However, increasing these parameters have a variety of other effects. For example, increasing the voltage and increasing the packet duration each increase muscle contraction, temperature rise and risk of electrical arcing. Increasing the fundamental frequency can lower the muscle contraction but it also reduces the treatment size. Likewise, increasing the number of packets increases the treatment size but also increases the temperature rise and the treatment delivery time. The treatment delivery time can be lowered by increasing the packet delivery rate, however, this increases the temperature rise. Therefore, managing the influences of various parameter changes is a complex endeavor. This is further complicated by the increments in which these changes are made. Once considering the number of parameters and increments in which they can be changed as related to the PEF waveform and dose itself, the set of combinations in staggering in volume. This is further compounded by the influence of electrode geometry, such as where a large electrode delivering a particular electrical voltage will have different characteristics than a smaller electrode, or monopolar versus bipolar and multipolar arrangements (and the separation distances between electrodes in these arrangements). These characteristics also include the temperature rise, treatment effect size, electrical current delivered, muscle contraction, electrical arcing risk, and time required to attain coverage of a targeted treatment effect size. Furthermore, certain conditioning solutions provided to the patient may specifically be used to target and reduce the induced muscle contraction. For instance, neuromuscular blockade with pancuronium bromide, vecuronium, succinylcholine, and other blockades may be used. This reduction in muscle contraction may be used to facilitate treatment doses with lower frequencies, longer packet durations, or higher voltages to attain larger treatment effects while maintaining acceptably safe muscle contractions.

It may be appreciated that parameters can be manipulated to reduce the probability of inducing an arrhythmia. Typically, high voltage energy can trigger a premature action potential within the cardiac muscle as the delivered energy increases the cardiac muscle cell membrane permeability allowing ion transport, which can induce cardiac arrhythmias, especially ventricular fibrillation. However, both shorter pulse durations (i.e. higher frequencies such as 100-600 kHz) and biphasic waveform shapes theoretically and practically reduce the probability of inducing an arrhythmia. Stimulation of excitable tissues follows a strength-duration curve. For example, not only does stimulation threshold increase with shorter pulse duration, but ventricular fibrillation induction threshold also increases significantly as pulse width decreases. Since the chronaxie for stimulation is more than an order of magnitude less than that of fibrillation, shorter pulses are much less likely to induce ventricular fibrillation. The exponential increase in amplitude to stimulate tissue as pulse width decreases results in 1-5 microsecond PEFs being much safer for the heart than those of 70-100 microseconds. Notably, the 70-100 microsecond pulses common in conventional IRE-type ablations at 3 kV would fall in the ventricular fibrillation induction range of these strength duration curves, while shorter microsecond pulses would fall above the stimulation threshold but below the ventricular fibrillation induction threshold for the same amplitude. Additionally, two other factors result in biphasic waveforms being less arrhythmogenic than monophasic waveforms: 1) biphasic waveforms have higher thresholds for stimulation and ventricular fibrillation induction than monophasic waveforms, and 2) biphasic waveforms result in more homogenous tissue polarization, which reduces the likelihood of post-shock voltage gradients initiating an arrhythmia.

However, since the induction of cardiac arrhythmia is highly dependent on the three-dimensional cardiac substrate (including cardiac fiber anisotropy), heterogeneous conductivity (including in tissue planes between the electrical source and the myocardium), and the complex spatiotemporal interaction of the electric field produced therapeutically with the intrinsic electrical wavefronts in the heart, an electrical therapy is evaluated in vivo to determine its true arrhythmogenic potential. Herein, the arrhythmogenic potential of the specialized PEF energy is assessed by testing it in a highly arrhythmogenic animal model and purposefully applying the therapy at the most vulnerable therapeutic anatomical locations and times within the cardiac rhythm.

### Materials and Methods

### Theoretical Evaluation of Arrhythmogenicity Likelihood

Numerical simulations offer a valuable tool for representing electrical tissue changes in response to PEF therapies. The cardiac safety of PEF therapies depends on several factors, including the electrical pulse duration, the applied voltage, and the distance to the heart. This analysis investigated these factors and their impact on two cardiac phenomena: cardiac activation and the induction of fibrillation. Comsol Multiphysics 5.4 (Comsol, Sweden) was used to model the voltage distribution and electric fields from various applied voltages.

### Geometry

The electrode was modeled as a series of five rings expanded within a 1 cm diameter airway. Each ring was a 1 mm width boundary in contact with the airway circumference and separated by 1 mm along the length of the airway. Epithelial and submucosal layers were considered 0.35 mm thick and cartilage 0.7 mm thick, consistent with approximate dimensions of porcine airway tissue layers. The airway region was placed within a 20 cm diameter and 40 cm long lung parenchymal region, with a 5 cm diameter dispersive plate electrode placed at the end.

### Tissue Electrical Properties

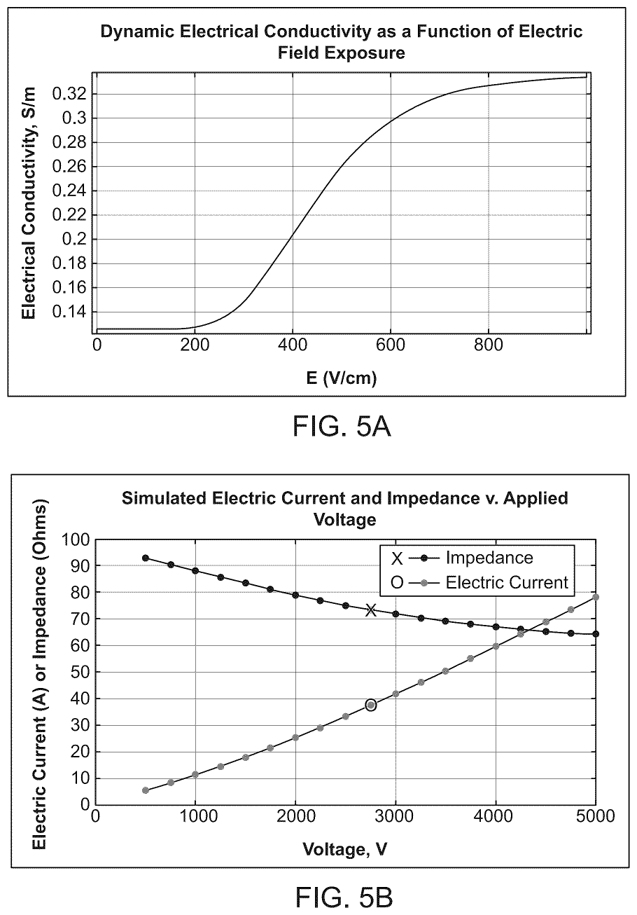

The airway interior was modeled as air, with an electrical conductivity of 1·10⁻⁷ S/m, while epithelial and cartilage layers each had electrical conductivity of 0.362 S/m. The lung parenchyma was considered to exhibit dynamic electrical conductivity as a function of electrical field, as described previously. This resulted in the electrical conductivity function of

$$σ ( E ) = {σ}_{0} + A \times {e}^{- {e}^{\frac{- ( E - B )}{C}}} \qquad ( 1 )$$

where σ₀ is the baseline electrical conductivity of inflated lung at a frequency consistent with 1 μs pulse duration (0.126 S/m), which increases with electric field exposure to approach the electrical conductivity of inflated lung at frequencies in the β-dispersion realm (200 MHz, 0.335 S/m). The 1 μs pulse duration was selected since it is within the typical range of biphasic PEF waveforms. The values for B and C provide the center and ± distance to the inflection points along the curve of 400 and 125 V/cm, respectively, which produces a conductivity function that increases as electric field exposure increases from approximately 250 to approximately 1000 V/cm (FIGS. 5A-5B). This range and curve shape is consistent with experimental data. Only electrical effects were modeled in the simulation, as this is the most influential in arrhythmia risk.

### Numerical Simulation Conditions

The geometry was meshed into 1.24.106 elements (extra fine Physics-controlled mesh). The external boundaries were considered electrically insulating, while interior boundaries followed current conservation. The dispersive pad boundaries were set to ground. The electrode rings were evaluated in a parametric study, with the applied electrical voltage evaluated from 1000 to 3000 V in 1000 V increments. The electrical voltage as a function of distance from the electrode rings in the tissue was determined.

### Theoretical Evaluation of Arrhythmogenicity

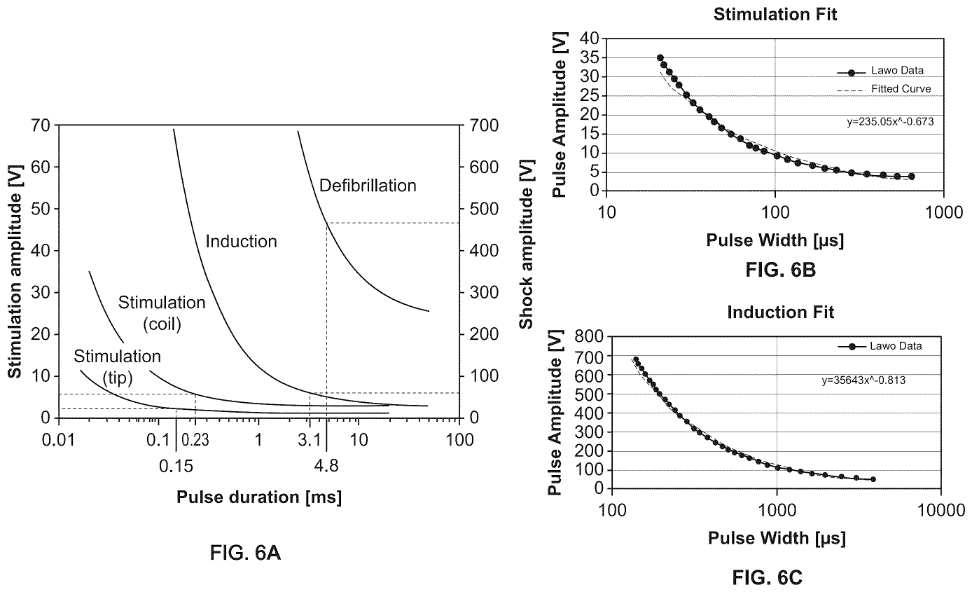

Both cardiac stimulation (i.e. pacing), and arrhythmia induction are voltage and pulse duration-dependent processes, and those strength-duration relationships were extracted from reference data that described them for approximately 90 μs-50 ms monophasic pulses. Notably, the thresholds to pace the heart and to induce arrhythmia diverge sharply below pulse widths of ˜1 ms. Data from monophasic pulses were extrapolated since the most robust PEF-related arrhythmia literature was conducted with monophasic pulses, and biphasic waveforms have been previously shown to have higher stimulation and VF induction thresholds, meaning monophasic pulses represent a worst-case scenario. The reference data were extrapolated by fitting the VF induction and stimulation curves with a power law relationship as depicted in FIGS. 6A-6C. These data were used to infer the likelihood of inducing either cardiac effect as a function of applied voltage and distance from the heart for various pulse durations between 1 and 100 μs.

### In Vivo Porcine Experiments

In this IACUC approved study, four swine (44.3-63.3 kg) received PEF energy. Electrocardiograms (ECGs) were recorded from a data acquisition module (DAQ) connected to an Ivy 7600 cardiac monitor (Ivy Biomedical, CT, USA) and the PEF generator. The cardiac monitor outputs the ECG waveform and a trigger pulse that corresponds in time to the R-wave of ventricular depolarization (the R-trigger), which allows precise control of PEF delivery timing relative to cardiac depolarization. The cardiac monitor was connected to the subject with a 4-lead cable. The DAQ concurrently recorded the ECG signals, R-triggers, and the delivery of PEF energy, enabling review of PEF energy delivery relative to the pig cardiac rhythm.

ECGs were recorded continuously during all experiments and were reviewed by a board-certified electrophysiologist to interpret any changes in cardiac rhythm or ECG waveform resulting from PEF delivery. ECGs were screened for signal saturation, an impact on R-R timing interval (heart rate), atrial activation or atrial arrhythmias, and ventricular activation or ventricular arrhythmias including tachycardia, bradycardia, and fibrillation. Any other ECG incidental findings were also noted.

C-arm fluoroscopy visualized the catheter position. Computed tomography (CT) images of the porcine chest were performed to determine airway-cardiac proximity.

### PEF System Background

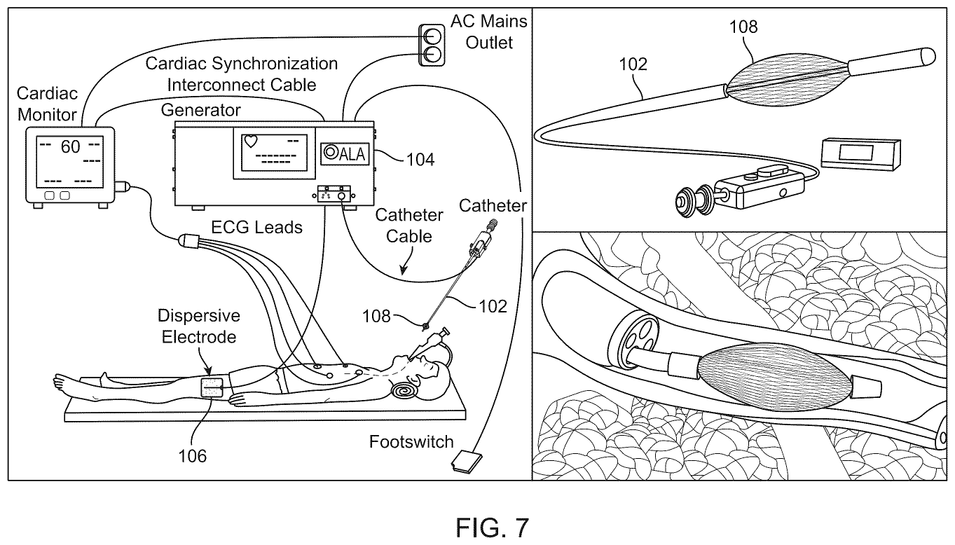

The system evaluated in this study uses a variety of PEF waveforms, including a specialized PEF used in treating chronic bronchitis. FIG. 7 illustrates an embodiment of the tissue modification system 100 that is used to deliver specialized PEF energy to target tissue in the lung, such as to treat chronic bronchitis. As illustrated, the monopolar system comprises a PEF generator 104, footswitch, energy delivery body 108 (e.g. expandable basket electrode), and a large dispersive electrode 106 placed at a distant location on the body. The generator delivers a series of packets of specialized PEF energy, with each packet comprised of multiple biphasic pulses. The basket electrode-tissue interface has markedly smaller surface area (0.67-1.30 cm², depending on airway diameter) relative to the dispersive electrode (approximately 100 cm³).

Three experiments were conducted to test worst-case timing of specialized PEF in directed attempts to deliberately induce cardiac arrhythmias: a single-packet of specialized PEF delivered at timed points over the entire cardiac cycle (Full ECG Sweep), a single-packet delivered at tightly timed intervals over the most vulnerable T-wave portion of the cardiac cycle (High-Resolution T-wave Delivery), and multiple packets arranged together and delivered over the entire cardiac cycle (Multiple Packets). All studies used a generator with custom software to allow for precise timing of energy delivery relative to the R-trigger and delivered specialized PEF energy in various positions in the airway tree, including distal and proximal sides of both the left and right lungs to account for positional and anatomical variability around the heart.

### Full ECG Sweep

In two pigs, a single packet of specialized PEF was delivered. The generator 104 sequentially extended the R-trigger delay interval before specialized PEF delivery in 25 ms increments. This resulted in specialized PEF delivery over all phases of the cardiac cycle. One packet was delivered every five heartbeats to allow observation of the cardiac response before the next packet delivery.

### High-Resolution T-Wave Delivery

In a third pig, a single packet was delivered at higher resolution (10 ms increments) to cover the entire duration of the anticipated vulnerable region of the T-wave. The T-wave is recognized as the portion of the ECG most susceptible to arrhythmia since the tissue at that time has differing degrees of ventricular repolarization, which can result in unidirectional block and arrhythmia induction when stimulated. One packet was delivered every five heartbeats to facilitate interpretation of the occurrence of changes to ECG and cardiac rhythm from the PEF treatment. The entire T-wave sweep was repeated at the clinical PEF dose, as well as a dose that delivered 44% more energy than the clinical dose.

### Multiple Packets

Most specialized PEF therapies deliver a bundle of packets to accumulate cellular injury and increase cell death beyond what would be produced by delivery of a single packet. To evaluate cardiac safety in the context of multiple packet deliveries, a fourth pig was used to investigate whether compounding PEF packets, delivered asynchronously, could induce arrhythmia when a single PEF packet could not.

The generator was set to deliver a sequence of 5 or 10 packets in a bundle. PEF bundle delivery was started at different times throughout the ECG waveform with a resolution of 40 ms to ensure adequate probing of all potentially vulnerable regions. Two trials had a resolution of 80 ms. The cadence of packets within each bundle was also varied, to deliver packets either quickly (5 Hz/300 ppm), slowly (0.66 Hz/40 ppm), or at a rate near the intrinsic heartrate of the animal (roughly 1.2 Hz/70 ppm). As with the prior experiments, ECGs were recorded for five beats following delivery of the bundle of packets to determine the presence of induced arrhythmias.

### Results

### Theoretical Evaluation

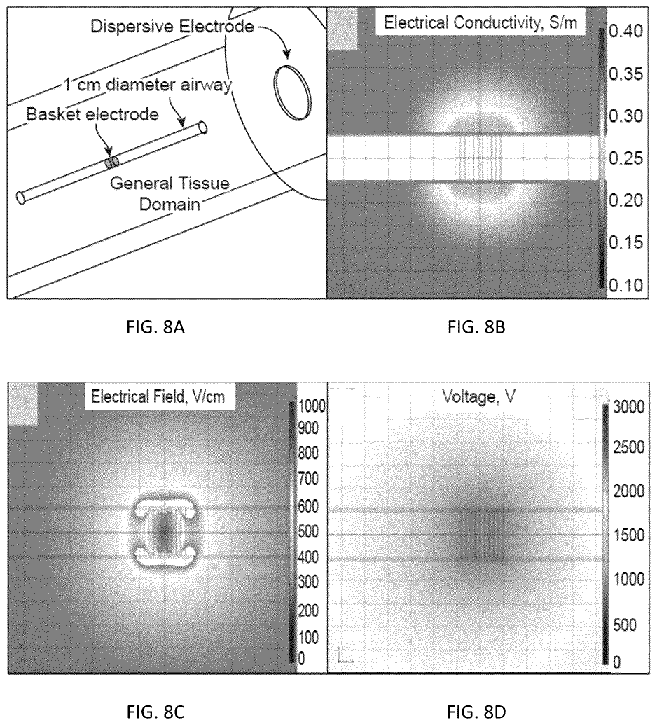

The geometry used in the numerical simulation, as well as representative distributions of electrical conductivity, electrical field, and electric voltage for a 3000 V pulse are shown in FIGS. 8A-8D.

Dynamic electrical conductivity invoked a subtle decrease in tissue impedance with higher applied voltages due to a greater volume of tissue experiencing a PEF-induced increase in electrical conductivity (FIGS. 5A-5B).

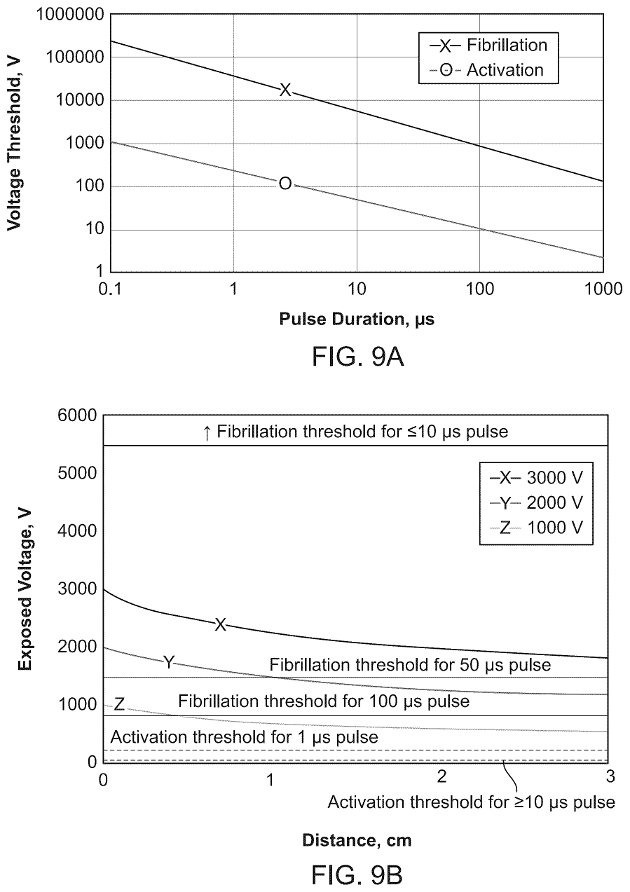

FIG. 9A shows how the extrapolated voltage thresholds for cardiac stimulation and fibrillation induction are separated by approximately two orders of magnitude, and decrease logarithmically with increasing monophasic pulse duration. Although activation energies for pulses greater than 1 μs are less than 250 V, the voltages required to induce fibrillation for 0.5, 1, 10, 50, and 100 μs duration are much higher, decreasing from 62.6 to 35.6, 5.48, 1.48, and 0.843 kV, respectively. This highlights the high sensitivity of cardiac arrythmia to pulse duration.

The data from the numerical simulations were combined with the extrapolated threshold curves to produce FIG. 9B. In FIG. 9B, electric voltage decay in tissue over 3 cm at varying applied voltages is overlaid with the thresholds for activation and fibrillation at different monophasic pulse durations. From FIG. 9B, all applied voltages exposed the tissue to activation energies out to 3 cm deep. Furthermore, the voltage to cause fibrillation for 50 and 100 μs duration pulses was exceeded within 1 cm for all pulses when the applied voltage was above 2 kV. Conversely, for pulse durations≤10 μs, none of the applied voltages resulted in tissue exposed to voltages capable of inducing fibrillation at any distance. These data indicate a substantial safety margin against cardiac arrhythmia induction for PEF treatments when the monophasic pulse duration is 10 μs or less.

### Experimental Evaluation

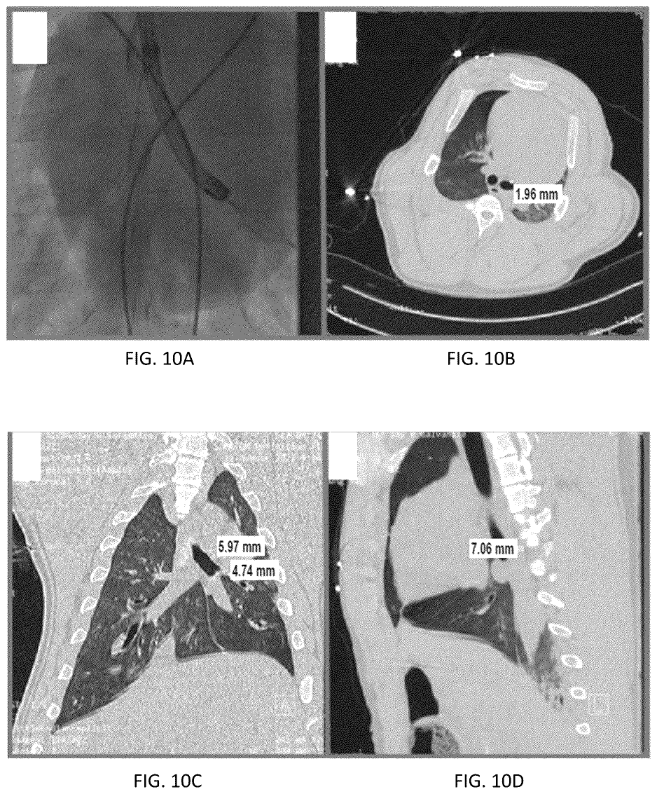

The minimum distance between the heart and mainstem bronchi where the basket electrode was placed was measured by combining a fluoroscopic image of basket deployment with CT images of the porcine chest indicated a distance as close as 2 mm (FIGS. 10A-10D). FIG. 10A illustrates a fluoroscopic image of bronchoscope in the left main bronchus of the swine with deployment of basket electrode. Representative swine CT scans are provided with proximity to the epicardial surface measured in the axial (FIG. 10B, 1.96 mm), coronal (FIG. 10C, 5.97 mm and 4.74 mm), and sagittal (FIG. 10D, 7.06 mm) planes.

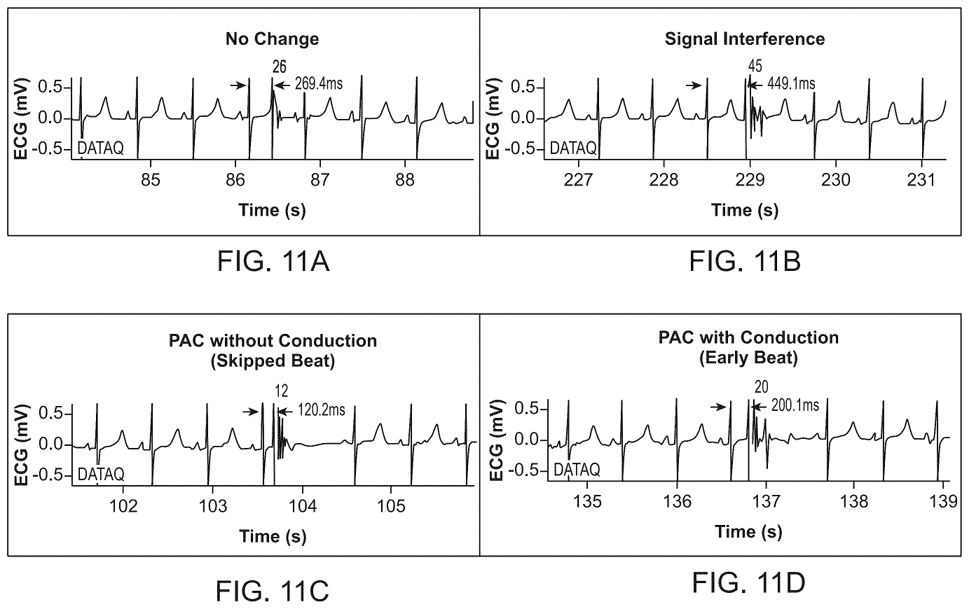

A total of 3.125 PEF packets were delivered to four pigs across the three studies. No sustained changes to the cardiac rhythm nor to the ECG waveform were noted, particularly no atrial nor ventricular fibrillation, no ST elevations, and no ventricular tachycardia. Overall, the cardiac response to the specialized PEF energy delivery fell into one of four categories, which are shown in FIGS. 11A-11D:
1) None (FIG. 11A): No effect or change to the ECG resulting from the packet delivery.
2) Signal Interference (FIG. 11B): specialized PEF delivery resulted in ECG artifact. When this artifact coincided with an ECG feature (e.g. QRS complex) the feature appearance was altered. There is no impact to cardiac activation or timing and is an artifact of the experimental setup and data recording devices.
3) Premature atrial contraction (PAC) without ventricular conduction (FIG. 11C): The delivered packet stimulates an atrial contraction prior to the native conducted beat from the sinoatrial (SA) node. The atrioventricular (AV) node is refractory during this premature beat, and thus the electrical signal does not conduct to capture the ventricles. The SA node, however, has been reset, and thus a subsequent normal heartbeat does not occur until a period following the premature atrial contraction that aligns with the basal heartrate. This manifests on an ECG as a ‘non-conducted.’ or skipped, beat with a cycle length approximately double the basal cycle length. The cardiac rhythm returns to the basal heartrate following the subsequent heartbeat.
4) Premature atrial contraction (PAC) with ventricular conduction (FIG. 11D): The delivered packet stimulates an atrial contraction prior to the native conducted beat from the SA node. The AV node is not refractory and permits the premature beat to conduct and activate the ventricles, resulting in normal ventricular conduction. This manifests as a premature heartbeat that occurs prior to when the basal heartbeat would be encountered. Following the premature heartbeat, the cardiac rhythm returns to its basal rate, which at times, is encountered with either sinus reset or with continued sinus timing.

The two PAC conditions are seen to alter that specific R-R interval but have no residual effect beyond the subsequent heartbeat. Arrows and numbers denote the time interval from QRS complex to PEF delivery. Bars and numbers denote when the PEF packet was delivered and the PEF packet number.

A summary of treatments delivered, and the adjudicated review of the cardiac rhythms and ECG waveforms, are provided in Table 2. There were no instances of sustained changes to the ECG waveform (e.g., ST-elevation) or to cardiac rhythm for any PEF deliveries, including no episodes of bradycardia, tachycardia, atrial fibrillation, or ventricle fibrillation. Furthermore, findings were limited to single PACs. This data is further delineated by lung and location in Table 2.

TABLE 2
Summary of PEF Delivery

| | R-Trigger | R-Trigger | Total | | | | | | | |
|---|---|---|---|---|---|---|---|---|---|---|
| | Delay | Delay | ECG | Total | Total PAC | Brady- | Tachy- | Atrial | Ventricle | ST |
| Focus | Range, ms | Resolution, ms | Sweeps | Packets | Observations | cardia | cardia | Fibrillation | Fibrillation | Elevation |
| ECG Sweep | 10-R* | 10 | 16 | 1174 | 186 | 0 | 0 | 0 | 0 | 0 |
| Specific T- | 100-490 | 10 | 4 | 160 | 46 | 0 | 0 | 0 | 0 | 0 |
| Wave Triggers | | | | | | | | | | |
| Multiple | 40-R* | 40 | 22 | 1610 | 71 | 0 | 0 | 0 | 0 | 0 |
| Packets | | | | | | | | | | |

*When complete ECG sweeps were performed, the R-wave trigger delay ranged from the shortest resolution timepoint until the subsequent next R-wave occurred. This varied based on pig heartrate.

Brief descriptions of the individual experiment results are provided below.

### Full ECG Sweep

A total of 1,180 packets were delivered across the two pigs. Each section (distal and proximal) of both the left and right lungs received a full cardiac cycle PEF sweep, resulting in 16 total complete cardiac cycle PEF sweeps. PACs occurred as described previously, and only with the basket electrode in the proximal mainstem bronchi. When a PAC occurred its impact was only on the timing of the immediate subsequent heartbeat, and this spontaneously resolved immediately in all cases, regardless of whether it was conducted to the ventricles or not. Overall, there were no safety risks, as defined by any arrhythmias, any aberrant ventricular conduction outside of the normal conducting system, or any changes to the ST segment, etc., associated with the packet delivery.

### High-Resolution T-Wave Delivery

During the high-resolution T-wave delivery, a single pig received 160 packets at the clinical PEF dose Overall, despite the high-resolution delivery of PEFs across the most-vulnerable portion of the ECG, no durable (>1 heartbeat) changes to cardiac rhythm were observed, and no appreciable safety risk associated with the biphasic PEF packet delivery in any region was noted.

### Multiple Packets

In all, 1,945 packets were delivered over 341 PEF activations to a single pig. PACs were noted, again only with the basket electrode in the proximal left mainstem bronchus when the treatment was delivered sufficiently beyond the refractory period. A summary of observations is provided in Table 2. Consistent with the two prior experiments, there were no observed incidences of durable changes to cardiac rhythm, including incidences of fibrillation or other dangerous arrhythmias. These findings were consistent regardless of the PEF packet delivery rate, whether 5 or 10 packets were delivered, and without regard for the initial timing of the first packet delivered. A discrete breakdown of the experimental conditions and PAC induction are in Table 3.

TABLE 3
Summary of experimental results for multi-
packet PEF delivery to the heart.

| | Total | | Delivery | | No | PAC without | PAC with |
|---|---|---|---|---|---|---|---|
| Set | Sets | Packets | Rate | | effect | conduction | conduction |
| 1 | 90 | 5 | 5 | Hz | 5 | 17 | 5 |
| 2 | 95 | 5 | ECG | | 59 | 5 | 10 |
| 3 | 89 | 5 | 0.66 | Hz | 32 | 1 | 21 |
| 4 | 29 | 10 | 5 | Hz | 0 | 6 | 5 |
| 5 | 9 | 10 | ECG | | 6 | 0 | 0 |
| 6 | 10 | 10 | 0.66 | Hz | 0 | 0 | 1 |

All animals treated in these studies had normal cardiac rhythms immediately post-treatment that lasted until their pre-specified survival timepoints. Therefore, these data demonstrate the absence of any clinically meaningful cardiac arrythmias regardless of test conditions for electrode location, PEF delivery relative to the cardiac rhythm, nor number of subsequent packets.

### Discussion

A systematic exploration of the arrhythmogenic potential of an embodiment of a biphasic, monopolar PEF system in the airways was performed. A numerical simulation evaluated the likelihood of arrhythmia induction as a function of pulse duration, applied voltage, and distance to the heart. Experimentally, individual packet dosing and waveform characteristics were matched to that of treatment parameters for clinical airway therapy for both single and multiple packet treatments.

No sustained changes to the ECG waveform or to the cardiac rhythm were observed despite the increasing vigor of the various attempts to induce an arrhythmia for the short-duration (≤10 μs) biphasic pulses tested in this experimental setup. These data are in stark contrast to results previously reported for a different PEF technology (i.e. irreversible electroporation) by Deodhar et al. In that study, 90 NanoKnife (AngioDynamics, NY, USA) pulses, each 70 μs pulse duration, were delivered over a range of voltages and locations relative to the heart. They observed a large proportion of the test conditions induced a range of cardiac arrhythmias, including ventricular tachycardia and ventricular fibrillation when energy was delivered asynchronously. When R-trigger cardiac synchronization was included according to their recommendations. ST elevation and T-wave inversions were still observed. These differences underscore that not all PEF technologies are alike, with key distinctions in terms of safety and efficacy for various electric field platforms and delivery systems. There are other differences between this study and the Deodhar study beyond pulse duration, including the electrode arrangement (monopolar here), and the biphasic waveform.

In this study the end effector was purposely placed in the closest airway positions to the heart (approximately 2 mm from cardiac muscle). Pigs are documented to be significantly more arrhythmogenic than humans. Despite these worst-case conditions, the only alteration to the ECG were occurrences of PACs when energy was delivered with the basket electrode in the proximal mainstem bronchi. The arrhythmogenicity of specialized PEF energy decreases dramatically for biphasic waveforms, such as the one used for the PEF therapy evaluated here.

Theoretical examination of cardiac activation and fibrillation thresholds demonstrated that activation is likely for short (<10 μs) individual monophasic pulses even at low PEF applied voltages (≤1 kV). However, fibrillation thresholds are approximately two orders of magnitude greater, and thus induction of arrhythmia was only noted when the pulse duration was 50 μs or longer, with 100 μs pulses able to induce fibrillation for all examined voltages. Notably, regarding the biphasic PEF waveforms described herein, the pulse duration for each phase is 0.5 to 5 μs, falls well within this ‘zone of safety’ for monopolar electrodes and applied voltages well above 3.0 kV. This is consistent with the conditions of the commercially available specialized PEF system experimentally evaluated for inducing arrhythmia in pigs. The system used a PEF protocol that encompassed voltage and frequency characteristics consistent with those evaluated in the simulation, and were shown to cause cardiac activation, but not fibrillation under any of the conditions tested.

One limitation of the theoretical portion of this study is that it evaluated arrhythmia risk for a single monophasic pulse. While this is consistent with some commercial PEF systems that use a series of long monophasic pulses (50-100 μs duration), it does not fully recapitulate the cardiac effects potentially induced by a biphasic waveform comprising a compilation of short pulses (0.5-5 μs) of alternating phase in rapid succession. This limitation is a result of the previous relevant explorations on activation and fibrillation risk for cardiac tissue as a function of pulse waveform characteristics. Additional theoretical work may better characterize the likelihood of activation and fibrillation induction under typical PEF ablation waveforms that incorporate these additional variables (biphasic pulse width, number of biphasic cycles comprising a complete packet, and delivery rate of multiple biphasic packets).

The specialized PEF therapy protocol evaluated experimentally is capable of therapeutic cell death. The protocol tested was optimized to produce significant epithelial (43±27%) and submucosal gland (3±4%) cell death without causing changes to airway integrity or function. The clinical utility of the protocol used by this system were evaluated in a multicenter clinical trial, where patients with chronic bronchitis (CB) were treated. No device-related adverse events were found, while patient-reported symptom improvements of −8.0 (p<0.001) and −14.7 (p<0.001) were observed for the chronic obstructive pulmonary disease assessment test (CAT) and St. George's Respiratory Questionnaire (SGRQ), respectively. These changes are well above the minimally clinically important differences in each test (−2 and −4 points, respectively). Tissue measures of goblet cell hyperplasia were reduced by roughly 39% (p<0.001) when comparing pretreatment with post-treatment airway tissue samples.

The occasional induction of PACs was the only noted cardiac impact from the delivery of specialized PEF in these studies and is consistent with the simulated prediction of cardiac stimulation but not arrhythmia induction. PACs are a common clinical phenomenon, occurring at least once per 24 hour period in >99% of the general adult population. The impact of the delivered energy was limited to only a single beat, never extending to any subsequent beats. Therefore, these findings do not demonstrate a measurable risk to patient health.

One consideration is that although the relatively healthy porcine hearts could be more resilient compared to human hearts with underlying disease states, such as myocardial ischemia. However, it should be noted that swine are nearly three times more sensitive electrically induced arrhythmia than humans, and thus serve as a highly sensitive model for cardiac safety. This study also relied on historic literature to demonstrate the arrhythmogenic potential of longer duration monophasic waveforms rather than a positive control. While it is possible that humans and pigs have different responses to monophasic versus biphasic waveforms, this is unlikely since both exhibit the same preferential response for biphasic versus monophasic cardioversion.

This study theoretically evaluated several relevant variables to cause arrhythmia, including applied voltage, pulse duration, and proximity to the heart. It experimentally evaluated a biphasic, monopolar. PEF technology for treating the airways to determine the safety of asynchronous PEF delivery throughout various regions of the ECG waveform. Despite the delivery of thousands of packets, including many directly over the vulnerable T-wave, only occasional PACs, and no cardiac arrhythmias or changes to ECG waveform were observed. This study demonstrates that the specialized PEF does not appear to require cardiac synchronization of energy delivery to prevent concerning arrhythmias.

By using a waveform that significantly reduces the chance of initiating arrhythmia (i.e. specialized PEF), energy can be delivered without cardiac synchronization. Consequently, energy can be delivered according to methods and procedures which were previously contraindicated.

In some embodiments, specialized PEF energy is delivered to target tissue throughout a heartbeat of the patient. In other embodiments, energy is delivered during specific periods of a heartbeat. A typical ECG trace includes a repeating cycle of a P wave representing atrial depolarization, a QRS complex representing ventricular depolarization and atrial repolarization, and a T wave representing ventricular repolarization. Typically, there is a portion of the heartbeat that is considered a “vulnerable period” of the cardiac muscle. Within one cardiac cycle (heartbeat), the vulnerable period of the ventricular muscle is denoted on an ECG by the entire T wave. The T-wave is recognized as the portion of the ECG that is most susceptible to arrhythmia since the tissue at that time has differing degrees of ventricular repolarization, which can result in unidirectional block and arrhythmia induction when stimulated. Typically, for ventricular myocardium, the vulnerable period coincides with the middle and terminal phases of the T wave. However, when high energy pulses are delivered in close proximity to the ventricle, the vulnerable period can occur several milliseconds earlier in the heartbeat. Therefore, the entire T wave can be considered to be within the vulnerable period of the ventricles. In some embodiments, energy (such as a single packet or a plurality of packets) is delivered throughout the vulnerable period of the ventricular muscle, throughout the T wave, through at least a portion of the T wave, through at least a phase of the T wave, through at least the middle phase of the T wave, through at least the terminal phase of the T wave, through at least a portion of the middle phase of the T wave and/or the terminal phase of the T wave, to name a few.

In some embodiments, specialized PEF energy is delivered to the target tissue by the generator wherein the generator sequentially extends an R-trigger delay interval before PEF delivery in 25 ms increments. This results in PEF delivery over all phases of the cardiac cycle. In other embodiments, a single packet is delivered at a higher resolution (10 ms increments) to cover the entire duration of the anticipated vulnerable region of the T-wave. In some instances. PEF therapies deliver a bundle of packets to accumulate cellular injury and increase cell death beyond what would be produced by delivery of a single packet. Thus, in some embodiments, a sequence, such as 5 or 10 packets in a bundle, are delivered during a vulnerable period. In some embodiments. PEF bundle delivery is started at different times throughout the ECG waveform with a resolution of 40-80 ms. In some instances, cadence of packet delivery within each bundle is varied to deliver packets either quickly (5 Hz/300 ppm), slowly (0.66 Hz/40 ppm), or at a rate near an intrinsic heartrate (roughly 1.2 Hz/70 ppm).

In some embodiments, specialized PEF is delivered during atrial contraction. Typically, delivery during atrial contraction is contraindicated due to an elevated risk of inducing an arrhythmia but such delivery can be achieved with the specialized PEF. Delivery during atrial contraction ensures that the heart is in the same physical state when energy is delivered during each dose. At the beginning of the cardiac cycle, both the atria and ventricles are relaxed (diastole). Blood is flowing into the right atrium from the superior and inferior vena cavae and the coronary sinus. Blood flows into the left atrium from the four pulmonary veins. The two atrioventricular valves, the tricuspid and mitral valves, are both open, so blood flows unimpeded from the atria and into the ventricles. Approximately 70-80 percent of ventricular filling occurs by this method. The two semilunar valves, the pulmonary and aortic valves, are closed, preventing backflow of blood into the right and left ventricles from the pulmonary trunk on the right and the aorta on the left. Contraction of the atria follows depolarization, represented by the P wave of the ECG. As the atrial muscles contract from the superior portion of the atria toward the atrioventricular septum, pressure rises within the atria and blood is pumped into the ventricles through the open atrioventricular (tricuspid, and mitral or bicuspid) valves. At the start of atrial systole, the ventricles are normally filled with approximately 70-80 percent of their capacity due to inflow during diastole. Atrial contraction, also referred to as the “atrial kick.” contributes the remaining 20-30 percent of filling. Atrial systole lasts approximately 100 ms and ends prior to ventricular systole, as the atrial muscle returns to diastole.

In some embodiments, specialized PEF is delivered during the maximum contraction peak of the atrial systole or ventricular systole. This is helpful when treating cardiac tissue wherein such delivery overcomes contact variability while the heart is beating. In another embodiment, specialized PEF is delivered when the myocardium starts the relaxation process after the maximum contraction. In such embodiments, the ratio of contact force electrode to tissue persists at the high range, which can be achieved by continuing to monitor the contact force or contact presence sensing. During this time, the relaxed myocardium presents a thinner conformation but the same energy is delivered. This facilitates the creation of a transmural lesion due to the higher energy per tissue thickness ratio.

Determining atrial systole or ventricular systole can be determined by contact force measurements, pressure monitoring (e.g. to synchronize the delivery of the pulses to the pressure, as a surrogate of the expected tissue contraction), and/or blood flow monitoring (e.g. using doppler ultrasound imaging to synchronize the delivery of the pulses with the flow as a surrogate of the expected contraction), to name a few.

In some embodiments, energy delivery is timed to provide artifact-free or artifact-reduced visualization of particular electrical features. It may be appreciated that when energy is delivered, the intracardiac electrical monitoring system typically presents a large electrical artifact on all surfaces of cardiac tissue. This artifact can make reading the surface ECG or intracardiac EGMs difficult for a brief period of time. The length of time depends on how quickly the recording amplifiers recover from saturation. Therefore, having restricted periods of delivery time may limit the ability of the user to visualize particular electrical features. However, the specialized PEF can be delivered unrestricted so delivery can be chosen for time periods that do not overlap with particular ECG and/or EGM features. This would allow improved or artifact-free visualization of these features. For example, energy delivery can be chosen to avoid the period of atrial depolarization which would allow for clearer visualization of atrial p-waves.

In some embodiments, such control over timing of energy delivery also improves compatibility with accessories and other systems. Some accessories or device systems, particularly patient-connected devices, provide functions that benefit from operating during periods without energy delivery. One such example is an electro-anatomical mapping system used when treating cardiac tissues, such as in the treatment of arrhythmias. It is preferred to avoid energy delivery to the tissue while electrical measurements are taken of the cardiac tissue. Thus, energy delivery can be timed to these preferred periods without the complications of additional syncing requirements to the heartbeat. This eliminates or reduces artifacts and mitigates potential for system damage.

In some embodiments, specialized PEF energy is delivered at a dose dependent rate. In some embodiments, a dose is a predetermined delivery of energy that results in a desired treatment effect, such as the formation of a lesion. In such instances, one dose creates one lesion. Since a dose is based on energy delivered, a dose can be delivered over various time periods. When unencumbered by cardiac synchronization, the delivery of specialized PEF energy can be tuned to the desired dose of the energy. In such instances, the energy is delivered in the shortest amount of time that provides the desired effect. It may be appreciated that a dose may include delays to allow for dissipation of heat, to reduce or eliminate any thermal effects. However, when accommodating both thermal effects and cardiac synchronization, doses are variable and can exceed clinically desirable treatment times. In some instances, delays arc manipulated so the dose fits within a desired clinical treatment time even when the outcome is not completely optimal for treatment outcome. For example, to accommodate patients having a variety of potential heartrates (e.g. 40-140 bpm) the dose is designed so as to deliver the energy fast enough so that accommodating 40 bpm does not take too long clinically but also to deliver the energy slow enough so that accommodating 140 bpm does not cause excessive temperature rise. But delivering a specialized PEF energy as desired without synchronization with the cardiac cycle allows the energy to be delivered dependent entirely on optimization of the dose, thus at a dose-dependent rate. Typically, this results in a faster overall treatment time while still allowing for delays, such as to reduce any thermal damage. This may be particularly useful for lower voltage doses that can be delivered faster without incurring extensive temperature rise.

In some embodiments, specialized PEF energy is delivered at a constant delivery rate which has the benefit of providing a constant time to form a lesion. When energy delivery is not constant, such as depending on the cardiac cycle, the delivery time is dependent on the heart rate. However, the average resting heart rate varies across age, sex, body mass index (BMI), and sleep habits. In some instances, daily resting heart rates differ between individuals by as much as 70 beats per minute (bpm). Men typically have a daily resting rate between 50 and 80 bpm, while women typically have a daily resting rate between 53 and 82 bpm. Men with somewhat average BMI tend to have the lowest resting heart rates, while people with very low or very high BMIs tended to have higher rates. There is also variability within a single patient. Compared to men, women of childbearing age showed greater variability in their individual resting rates. But for all patients, there is even a small seasonal change. The average daily resting heart rate in both men and women peaks in early January, before declining to a yearly low at the end of July. All of this introduces an unpredictability in the time it takes to form a lesion in any given patient when syncing the energy delivery to the heartbeat. However, if such synching is eliminated by the use of the specialized PEF, a constant delivery rate can be used which will translate to a constant time to form a lesion. This is particularly advantageous in the treatment of cardiac tissues for electrophysiologists who deliver energy with the use of a “dragging” technique. In such a technique, the delivery electrode is dragged over the cardiac tissue at a constant rate to deliver the energy, rather than placing the delivery electrode at one location while delivering the energy. By delivering the energy at a constant rate, the energy is uniformly delivered to the cardiac tissue. This is also advantageous for automated systems, such as robotic surgery, which are often predetermined or have limited ability to provide variation or adaptation.

Once energy delivery is disassociated from syncing to the heartbeat, energy delivery can be associated with other anatomy based features. For example, energy delivery can be synced with respiration. Respiratory expansion may be detected by use of, for example, a thoracic belt, bellows, or cushion. The belt should be placed around the lower chest for “chest breathers” and around the mid abdomen for “abdominal breathers”. Impedance plesmythography devices that measure changes in electrical resistance across the chest with respiration may alternatively be used. This may be particularly useful when treating lung tissue or ablating tumors in or around the lungs. Thus, energy delivery may be actuated when the lung is not in motion which may allow for more consistent and/or predictable energy delivery. This also allows for avoidance of energy delivery during particularly disruptive events, such as coughing or sneezing. Likewise, when delivering energy near coronary vasculature or other parts of the vasculature, delivery can be tuned to reduce vasospasm risk. For example, when delivering energy to tissue near coronary vessels, the delivery rate may be reduced so as to avoid or reduce smooth muscle contraction of the vessels.

Once energy delivery is disassociated from syncing to the heartbeat, energy delivery can alternatively be actuated by feedback control, unencumbered by syncing with the heartbeat. Examples of feedback control include temperature monitoring, impedance monitoring, pH monitoring, contact detection and/or contact force, to name a few. Temperature monitoring can be utilized to more easily and effectively keep thermal damage at a minimal or non-present level. PEF energy is considered “non-thermal” because the tissue receiving the energy does not suffer thermal injury or damage. This is typically achieved by the incorporation of predetermined timing delays in the waveform which mitigate temperature rise. These delays essentially slow down the procedure. Likewise, built-in safety margins result in longer procedures than necessary. Real-time temperature monitoring shifts the temperature mitigation away from pre-determined delays, making the energy delivery more efficient. This results in faster treatments while keeping thermal damage reduced or eliminated.

In some embodiments, energy delivery is actuated by impedance-based feedback control. It may be appreciated that measured impedance may vary depending on the position of the energy delivery electrode. When the delivery electrode is buried in tissue (i.e. “encapsulated”), the impedance will be higher than merely touching the tissue, which in turn is higher than in blood not touching tissue. Too much encapsulation can result in greater thermal damage relative to PEF treatment. Too low an impedance can result in delivery primarily into blood rather than tissue leading to insufficient or non-existent lesion formation. Thus, in some embodiments, energy delivery is actuated when impedance is in a desired range reflecting desired encapsulation in tissue.

In some embodiments, energy delivery is actuated by feedback from one or more sensors configured to detect distribution of an agent within the body, such as within a particular region of the body or particular tissue(s). This may be useful when the procedure involves gene therapy, electrochemotherapy or delivery of the agent to cells within the body. In some embodiments, the agent comprises molecules and a key feature of molecule transfer in the body involves biodistribution of the molecules. Example molecules include plasmids. DNA plasmids. RNAs (e.g. messenger RNA (mRNA), small interfering RNA (siRNA), micro RNA), oligonucleotides, antisense oligonucleotides (ASO), proteins and/or materials which invoke genetic or epigenetic changes in the cellular behavior, to name a few. In order to successfully transfer the molecules to the cells, the molecules are to be in a desired location within the body at a desired concentration at a desired time in relation to the delivery of the energy for transfer. Thus, when these critical factors coincide, energy is able to be delivered regardless of the cardiac cycle. This provides increased effectiveness and efficiency.

In one embodiment, one or more impedance sensors may be utilized to measure the impedance between, for example, two tines or between a tine and central shaft on a delivery device, wherein agent is delivered through one or more tines. If the injected agent has different conductivity than the surrounding tissue the sensor may provide information about the distribution of the agent. In another embodiment, a pH, fiber optic, bipolar impedance, or other type of sensor may be utilized to detect when the agent spread, for example, from a central injection to a lateral tine. In some embodiments, the agent is a drug loaded with a radiopaque dye. In such instances, imaging is utilized rather than a sensor to provide feedback control for the PEF energy delivery.

These alternative delivery options provide better outcomes but also increase the speed of the procedure. Since portions of time that are typically excluded from energy delivery are now available, including constant energy delivery for any given period of time, more energy can be delivered in a shorter amount of time. Typically, when accounting for cardiac synchronization, only 20% of the available time (e.g. the whole cardiac cycle) may be utilized for energy delivery. By eliminating these restrictions with the use of specialized PEF, the remaining 80% is now available. This is a five-fold increase or an increase of 500%. Likewise, when multi-spline or multi-tined devices are used for energy delivery, the individual energy delivery elements (e.g. splines, tines, etc.) can be used sequentially or in any desired order without regard for the cardiac state at any given time. This allows the sequence or pattern of activations to be closer to together over time leading to increased energy delivery rate and decreased procedure time.

Additional example embodiments of energy delivery systems configured to provide PEF therapy to various parts of the body are provided in international patent application number PCT/US2021/044469 titled “PULSED ELECTRIC FIELD TRANSFER OF MOLECULES TO CELLS WHILE IN THE BODY” filed Aug. 3, 2021, international patent application number PCT/US2022/019719 titled “DEVICES FOR THE DELIVERY OF PULSED ELECTRIC FIELDS IN THE TREATMENT OF CARDIAC TISSUE” filed Mar. 10, 2022, and international patent application number PCT/US2022/044021 titled “CONTROLLED LESION AND IMMUNE RESPONSE TO PULSED ELECTRIC FIELD THERAPY” filed Sep. 19, 2022, all of which are incorporated herein by reference for all purposes. Likewise, a variety of forms of PEF energy may be used such as those described in the references incorporated herein and according to international patent application number PCT/US2021/026221 titled “PULSED ELECTRIC FIELD WAVEFORM MANIPULATION AND USE”, filed Apr. 8, 2021, all of which are incorporated by reference for all purposes.

As mentioned previously, one or more energy delivery algorithms 152 are programmable, or can be pre-programmed, into the generator 104 for delivery to the patient P. The one or more energy delivery algorithms 152 specify electric signals which provide the energy. It may be appreciated that a variety of energy delivery algorithms 152 may be used. In some embodiments, the algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, and the fundamental frequency of the pulse sequence, to name a few. Additional parameters may include switch time between polarities in biphasic pulses, dead time between biphasic cycles, and rest time between packets, which will be described in more detail in later sections. There may be a fixed rest period between packets, or packets may be gated to a particular metric. There may be a deliberate, varying rest period algorithm or no rest period may also be applied between packets. A feedback loop based on sensor information and an auto-shutoff specification, and/or the like, may be included.

### A. Voltage

The voltages used and considered may be the tops of square-waveforms, may be the peaks in sinusoidal or sawtooth waveforms, or may be the RMS voltage of sinusoidal or sawtooth waveforms. In some embodiments, the energy is delivered in a monopolar fashion and each high voltage pulse or the set voltage 416 is between about 500 V to 10,000 V, particularly about 3500 V to 4000 V, about 3500 V to 5000 V, about 3500 V to 6000 V, including all values and subranges in between including about 250 V, 500 V, 1000 V, 1500 V, 2000V, 2500 V, 3000 V, 3500 V, 4000 V, 4500 V, 5000 V, 5500 V, 6000 V to name a few. Voltages delivered to the tissue may be based on the setpoint on the generator 104 while either taking in to account the electrical losses along the length of the device 102 due to inherent impedance of the device 102 or not taking in to account the losses along the length, i.e., delivered voltages can be measured at the generator or at the tip of the instrument.

It may be appreciated that in various embodiments the output is controlled or modified to achieve a desired current rather than voltage. In some embodiments, the energy is delivered in a monopolar fashion and has a current of 20 amps, 21 amps, 22 amps, 23, amps, 24 amps, 25 amps, 26 amps, 27 amps, 28 amps, 29 amps, 30 amps, 31 amps, 32 amps, 33 amps, 34 amps or 35 amps to name a few.

### B. Frequency

It may be appreciated that the number of biphasic cycles per second of time is the frequency when a signal is continuous. Typically, the signal has a frequency in the range 100-600 kHz, such as 100-200 kHz, 100-300 kHz, 200-400 kHz, 200-500 kHz, 300-400 kHz, 300-500 kHz, 300-600 kHz, 400-500 kHz, 400-600 kHz, 500-600 kHz, 100 kHz, 200 kHz, 300 kHz, 400 kHz, 500 kHz, or 600 kHz, to name a few.

### C. Voltage-Frequency Balancing

The frequency of the waveform delivered may vary relative to the treatment voltage in synchrony to retain adequate treatment effect. Such synergistic changes would include the decrease in frequency, which evokes a stronger effect, combined with a decrease in voltage, which evokes a weaker effect. For instance, in some cases the treatment may be delivered using 3000 V in a monopolar fashion with a waveform frequency of 600 kHz, while in other cases the treatment may be delivered using 2000 V with a waveform frequency of 400 kHz.

### D. Packets

As mentioned, the algorithm 152 typically prescribes a signal having a waveform comprising a series of energy packets wherein each energy packet comprises a series of high voltage pulses. The cycle count 420 is half the number of pulses within each biphasic packet. Referring to FIG. 4, the first packet 402 has a cycle count 420 of two (i.e. four biphasic pulses). In some embodiments, the cycle count 420 is set between 2 and 1000 per packet, including all values and subranges in between. In some embodiments, the cycle count 420 is 5-1000 per packet, 2-10 per packet, 2-20 per packet, 2-25 per packet, 10-20 per packet, 20 per packet, 20-30 per packet, 25 per packet, 20-40 per packet, 30 per packet, 30-45 per packet, 45 per packet, 20-50 per packet, 30-60 per packet, up to 60 per packet, up to 80 per packet, up to 100 per packet, up to 1,000 per packet or up to 2,000 per packet, including all values and subranges in between.

The packet duration is determined by the cycle count, among other factors. For a matching pulse duration (or sequence of positive and negative pulse durations for biphasic waveforms), the higher the cycle count, the longer the packet duration and the larger the quantity of energy delivered. In some embodiments, packet durations are in the range of approximately 50 to 1000 microseconds, such as 50 μs, 60 μs, 70 μs, 80 μs, 90 μs, 100 μs, 125 μs, 150 μs, 175 μs, 200 μs, 250 μs, 100 to 250 μs, 150 to 250 μs, 200 to 250 μs, 500 to 1000 μs to name a few. In other embodiments, the packet durations are in the range of approximately 100 to 1000 microseconds, such as 150 μs, 200 μs, 250 μs, 500 μs, or 1000 μs.

The number of packets delivered during treatment, or packet count, typically includes 1 to 250 packets including all values and subranges in between. In some embodiments, the number of packets delivered during treatment comprises 2-5 packets, 3 packets, 5 packets, 5-10 packets, 10 packets, 12 packets, 10-15 packets, 15 packets, 20 packets, 15-20 packets, 25 packets, 30 packets or greater than 30 packets

### E. Rest Period

In some embodiments, the time between packets, referred to as the rest period 406, is set between about 0.001 seconds and about 5 seconds, including all values and subranges in between. In other embodiments, the rest period 406 ranges from about 0.01-0.1 seconds, including all values and subranges in between. In some embodiments, the rest period 406 is approximately 0.5 ms-500 ms, 1-250 ms, or 10-100 ms to name a few.

### F. Batches

In some embodiments, the signal is synced with the physiological or other features so that each packet is delivered synchronously within a designated period or in response to a designated trigger. It may be appreciated that the packets that are delivered at such times may be considered a batch or bundle. Thus, each batch has a desired number of packets so that at the end of a treatment period, the total desired number of packets have been delivered. Each batch may have the same number of packets, however in some embodiments, batches have varying numbers of packets.

In some embodiments, only one packet is delivered upon trigger. In such instances, the rest period may be considered the same as the period between batches. However, when more than one packet is delivered between batches, the rest time is typically different than the period between batches. In such instances, the rest time is typically much smaller than the period between batches. In some embodiments, each batch includes 1-10 packets, 1-5 packets, 1-4 packets, 1-3 packets, 2-3 packets, 2 packets, 3 packets, 4 packets 5 packets, 5-10 packets, to name a few. In some embodiments, each batch has a period of 0.5 ms-1 sec, 1 ms-1 sec, 10 ms-1 sec, 10 ms-100 ms, to name a few. In some embodiments, the period between batches is variable. In some instances, the period between batches is 0.25-5 seconds.

Treatment of a tissue area ensues until a desired number of batches are delivered to the tissue area. In some embodiments, 2-50 batches are delivered per treatment, wherein a treatment is considered treatment of a particular tissue area. In other embodiments, treatments include 5-40 batches, 5-30 batches, 5-20 batches, 5-10 batches, 5 batches, 6 batches, 7 batches, 8 batches, 9 batches, 10 batches, 10-15 batches, etc.

### G. Switch Time and Dead Time

A switch time, also known as inter-phase delay, is a delay or period of no energy that is delivered between the positive and negative peaks of a biphasic pulse, as illustrated in FIG. 4. In some embodiments, the switch time ranges between about 0 to about 1 microsecond, including all values and subranges in between. In other embodiments, the switch time ranges between 1 and 20 microseconds, including all values and subranges in between. In other embodiments, the switch time ranges between about 2 to about 8 microsecond, including all values and subranges in between.

Delays may also be interjected between each biphasic cycle, referred as “dead-time” or inter-pulse delay. Dead time occurs within a packet, but between biphasic pulses. This is in contrast to rest periods or inter-packet delays which occur between packets. In other embodiments, the dead time 412 is in a range of approximately 0 to 0.5 microseconds, 0 to 10 microseconds, 2 to 5 microseconds, 0 to 20 microseconds, about 0 to about 100 microseconds, or about 0 to about 100 milliseconds, including all values and subranges in between. In some embodiments, the dead time 412 is in the range of 0.2 to 0.3 microseconds. Dead time may also be used to define a period between separate, monophasic, pulses within a packet.

Delays, such as switch times and dead times, are introduced to a packet to reduce the effects of biphasic cancellation within the waveform. In some instances, the switch time and dead time are both increased together to strengthen the effect. In other instances, only switch time or only dead time are increased to induce this effect.

### G. Waveforms

It may be appreciated that in some embodiments the waveform 400 has symmetric pulses such that the voltage and duration of pulse in one direction (i.e., positive or negative) is equal to the voltage and duration of pulse in the other direction. It may be appreciated that in other embodiments the waveform 400 has voltage imbalance. For example, each packet 402, 404 may be comprised of a first biphasic cycle (comprising a first positive pulse peak 408 having a first voltage V1 and a first negative pulse peak 410 having a second voltage V2) and a second biphasic cycle (comprising a second positive pulse peak 408′ having first voltage V1 and a second negative pulse peak 410′ having a second voltage V2). Here the first voltage V1 is greater than the second voltage V2. The first and second biphasic cycles are separated by dead time 412 between each pulse. Thus, the voltage in one direction (i.e., positive or negative) is greater than the voltage in the other direction so that the area under the positive portion of the curve does not equal the area under the negative portion of the curve. This unbalanced waveform may result in a more pronounced treatment effect. It may be appreciated that in some embodiments, imbalance includes pulses having pulse widths of unequal duration. In some embodiments, the biphasic waveform is unbalanced, such that the voltage in one direction is equal to the voltage in the other direction, but the duration of one direction (i.e., positive or negative) is greater than the duration of the other direction, so that the area under the curve of the positive portion of the waveform does not equal the area under the negative portion of the waveform.

It may be appreciated that the devices, systems and methods described herein deliver energy to the target tissue with an energy delivery system. Generally, the energy delivery systems include a specialized energy delivery device, a waveform generator and at least one distinct energy delivery algorithm. Additional accessories and equipment may be utilized. For example, in some embodiments, the energy delivery device is delivered through an endoscope, typically specific to the anatomical location to which it is being used, such as gastroscopes (upper GI endoscopy, which includes the stomach, esophagus, and small intestine (duodenum)), colonoscopes (large intestine), bronchoscopes (lungs), laryngoscopes (larynx), cystoscopes (urinary tract), duodenoscopes (small intestine), enteroscopes (digestive system), ureteroscopes (ureter), hysteroscopes (cervix, uterus), etc. It may be appreciated that in other embodiments, the energy deliver device is deliverable through a catheter, sheath, introducer, needle or other delivery system.

Endoluminal access allows treatment of target tissue from within various lumens in the body. Lumens are the spaces inside of tubular-shaped or hollow structures within the body and include passageways, canals, ducts and cavities to name a few. Example luminal structures include blood vessels, esophagus, stomach, small and large intestines, colon, bladder, urethra, urinary collecting ducts, uterus, vagina, fallopian tubes, ureters, kidneys, renal tubules, spinal canal, spinal cord, and others throughout the body, as well as structures within and including such organs as the lung, heart and kidneys, to name a few. In some embodiments, the target tissue is accessed via the nearby luminal structure. In some instances, an energy delivery device is advanced through various luminal structures or branches of a luminal system to reach the target tissue location. For example, when accessing a target tissue site via a blood vessel, the energy delivery device may be inserted remotely and advanced through various branches of the vasculature to reach the target site. Likewise, if the luminal structure originates in a natural orifice, such as the nose, mouth, urethra or rectum, entry may occur through the natural orifice and the energy delivery device is then advanced through the branches of the luminal system to reach the target tissue location. Alternatively, a luminal structure may be entered near the target tissue via cut-down or other methods. This may be the case when accessing luminal structures that are not part of a large system or that are difficult to access otherwise.

It may be appreciated that a variety of anatomical locations may be treated endoluminally with the systems and methods described herein. Examples include luminal structures themselves, soft tissues throughout the body located near luminal structures and solid organs accessible from luminal structures, including but not limited to liver, pancreas, gall bladder, kidney, prostate, ovary, lymph nodes and lymphatic drainage ducts, underlying musculature, bony tissue, brain, eyes, thyroid, etc. It may also be appreciated that a variety of tissue locations can be accessed percutaneously or by other methods.

The energy delivery device delivers energy provided by the waveform generator according to the at least one distinct energy delivery algorithm. It may be appreciated that, in some embodiments, the energy delivery device also delivers an agent. However, in other embodiments, the agent is delivered by a separate device, such as by IV, catheter, or needle injection. Optionally, the agent may be delivered both by the energy delivery device and by a separate device. Example embodiments of specialized energy delivery devices are provided herein primarily focused on monopolar energy delivery, however, it may be appreciated that bipolar or multi-polar arrangements may be used.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), cither with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for delivering energy to a target tissue within a torso of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein the energy has a waveform comprising a plurality of biphasic pulses each having a voltage of at least 1000V and each is below a threshold for induction of arrhythmia, and wherein energy delivery is disassociated from syncing to the cardiac cycle and is actuated by feedback control.

2-3. (canceled)

4. The system of claim 1, wherein the pulses are spaced so that at least one of the pulses is delivered to the target tissue during at least a portion of a T-wave of the cardiac cycle.

5-7. (canceled)

8. The system of claim 1, wherein the plurality of pulses comprises at least one packet of pulses.

9. The system of claim 8, wherein the at least one packet of pulses comprises at least six packets of pulses, wherein each packet includes 40 biphasic pulses with inter-pulse delays of 1000 microseconds.

10. The system of claim 1, wherein each pulse has a pulse duration of less than or equal to 10 μs.

11. The system of claim 10, wherein each pulse has a pulse duration in a range of 0.5 μs to 5 μs.

12-15. (canceled)

16. The system of claim 1, wherein the dose has a delivery time of at least the cardiac cycle.

17-19. (canceled)

20. The system of claim 1, wherein the target tissue comprises a tumor and the energy delivery body comprises a probe.

21. (canceled)

22. The system of claim 1, further comprising a robotic apparatus for manipulating the energy delivery body within the patient.

23. The system of claim 1, wherein energy delivery is synced with respiration of the patient without cardiac synchronization.

24. The system of claim 23, wherein the energy delivery is actuated when the lung is not in motion.

25. (canceled)

26. The system of claim 1, wherein the feedback control comprises temperature monitoring, impedance monitoring, pH monitoring, contact detection and/or contact force.

27. The system of claim 1, further comprising one or more sensors configured to detect an agent within the body and wherein energy delivery is actuated by feedback from the one or more sensors.

28-30. (canceled)

31. A system for delivering energy to a target tissue within a torso and not on or in a heart of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein the dose is comprised of a plurality of biphasic pulses, wherein at least one of the pulses is arranged within the dose so as to be received by the target tissue during a T-wave of the cardiac cycle and wherein the dose does not induce arrhythmia.

32. (canceled)

33. The system of claim 31, wherein the pulses each have a voltage is in a range of 1000 to 10,000V.

34. (canceled)

35. The system of claim 31, wherein the plurality of biphasic pulses comprises at least one packet of pulses.

36. The system of claim 35, wherein the at least one packet of pulses comprises at least six packets of pulses, wherein each packet includes 40 biphasic pulses with inter-pulse delays of 1000 microseconds.

37. The system of claim 31, wherein each pulse has a pulse duration of less than or equal to 10 μs.

38. The system of claim 37, wherein each pulse has a pulse duration in a range of 0.5 μs to 5 μs.

39-43. (canceled)

44. The system of claim 31, wherein the target tissue resides within a lung of the patient.

45. The system of claim 31, wherein the target tissue comprises resides within a gastrointestinal system, urinary or reproductive system of the patient.

46. (canceled)

47. The system of claim 31, wherein the target tissue comprises a tumor and the energy delivery body comprises a probe.

48. (canceled)

49. The system of claim 31, further comprising a robotic apparatus for manipulating the energy delivery body within the patient.

50-51. (canceled)

52. A system for delivering energy to a target tissue within a torso of a patient having a cardiac cycle comprising:
at least one energy delivery body configured to deliver energy to the target tissue; and
a generator in electrical communication with the at least one energy delivery electrode, wherein the generator includes an algorithm for delivering a dose of the energy to the at least one energy delivery electrode so that the dose is delivered to the target tissue, wherein the dose is comprised of a plurality of pulses and wherein energy delivery is synced with respiration of the patient without cardiac synchronization.

53-54. (canceled)
